(19) United States
(12) Patent Application Publication
(10) Pub. No.: US 2024/0288374 A1
(43) Pub. Date: Aug. 29, 2024

(54) PLASMONIC NANOPARTICLE PLATFORM FOR ANALYTE DETECTION

(71) Applicant: ZYMERON CORPORATION, Research Triangle Park, NC (US)

(72) Inventors: Guandong ZHANG, Cary, NC (US); Zhiguo ZHOU, Chapel Hill, NC (US); Fang LIU, Cary, NC (US)

(21) Appl. No.: 18/538,408

(22) Filed: Dec. 13, 2023

Related U.S. Application Data
(60) Provisional application No. 63/484,683, filed on Feb. 13, 2023.

(51) Int. Cl. G01N 21/78

(52) U.S. Cl. CPC G01N 21/78

(57) ABSTRACT

Described herein are compositions and methods for the rapid detection of target analytes on surfaces and in solutions using colorimetric analysis. In some embodiments, the compositions and methods may include plasmonic nanoparticles, a binding receptor conjugated or adsorbed to a surface of the nanoparticles, and a polymer-ligand conjugate. In some embodiments, the target analytes may include one or more opioids.

### CROSS-REFERENCE TO RELATED APPLICATIONS

This application claims priority to U.S. Provisional Patent Application No. 63/484,683, filed on Feb. 13, 2023, which is incorporated by reference herein in its entirety.

### FEDERALLY SPONSORED RESEARCH

This invention was made with government support under contract number W911SR21C0025 awarded by the Department of Defense. The government has certain rights in the invention.

### REFERENCE TO SEQUENCE LISTING

This application was filed with a Sequence Listing XML in ST.26 XML format accordance with 37 C.F.R. § 1.831 at PCT Rule 13ter. The Sequence Listing XML file submitted in the USPTO Patent Center, “221987-0003-US02_sequence_listing_xml_22-NOV-2023.xml,” was created on Nov. 22, 2023, contains 1 sequence, has a file size of 2.05 Kbytes, and is incorporated by reference in its entirety into the specification.

### TECHNICAL FIELD

Described herein are compositions and methods for the rapid detection of target analytes on surfaces and in solutions using colorimetric analysis. In some embodiments, the compositions and methods may include plasmonic nanoparticles, a binding receptor conjugated or adsorbed to a surface of the nanoparticles, and a polymer-ligand conjugate. In some embodiments, the target analytes may include one or more opioids.

### BACKGROUND

Simple and convenient technologies for the identification of chemical and biological species are of great significance in environmental monitoring, public health, and disease diagnosis. However, rapid and cost-effective detection of medical compounds and biological species with high sensitivity and specificity is challenging. Traditional sensing techniques, such as chromatography, mass spectrometry, electrochemistry, field-effect transistors, surface plasmonic resonance sensors, and microgravimetric methods often require relatively expensive, cumbersome instruments, and a skilled professional to operate.

Colorimetric analysis is a method of determining the existence or presence and concentration of a chemical elements, chemical compounds, and small molecules in a solution with the assistance of a color reagent. The method is widely used in medical and chemical laboratories, as well as in industrial analysis. Besides the chemical color reagents, the use of plasmonic nanoparticles is widespread in colorimetric assays because of their simple, cost-effective fabrication, and simple processing, especially for gold nanoparticles (GNPs). GNPs colorimetric sensing response is a visual change in color, which allows easy interpretation of results. The surface plasmon resonance property makes GNPs sensitive to the surface status and the interparticle distance. In the interaction of GNPs (bare nanoparticles or surface functionalized) with analytes, aggregate and non-aggregate mode of nanoparticles changes and finally the change in the color of the solution is seen with naked eye without the need for advanced and costly equipment, indicating the presence or absence of target analytes. This change of mode and the subsequent color change is the basis of colorimetric detection of the plasmonic GNPs in detection of various analytes, include ions, chemical compounds, nucleic acid, proteins, peptides, and enzymes.

In plasmonic nanoparticle based colorimetric detection, popular approaches are based on cross-linking strategies. In most of the work, GNP aggregation is induced by the controlled assembly of ligand-functionalized GNPs with the formation of intermolecular bonds, which overpower the interparticle repulsive forces. Ligands such as peptides, nucleic acids, aptamers, and antibodies have been widely used to stabilize and functionalize GNPs for the development of colorimetric immunosensors. When target compounds are present in the detection system, the ligands bind to the targets resulting in the aggregation of the GNPs. However, the current plasmonic nanoparticle-based colorimetric detection has many challenges in the biological and medical applications. There is a lack of strategies to establish a standard platform which has broad applicability to measure the majority of biological and biomedical compounds.

What is needed are compositions and methods for the rapid detection of analytes on surfaces and in solutions using a simple color change.

### SUMMARY

One embodiment described herein is a system for colorimetric detection of a target analyte, the system comprising: plasmonic gold nanoparticles (GNPs); a binding receptor conjugated or adsorbed to a surface of the GNPs; and a polymer-ligand conjugate. In one aspect, the binding receptor is an opioid-binding receptor. In another aspect, the opioid-binding receptor is a water-soluble mu-opioid receptor (wsMOR). In another aspect, the target analyte is selected from acetorphine; acetyl fentanyl; acetyldihydrocodeine; acetylmethadol; acetyl-α-methylfentanyl; acryl fentanyl; AH-7921; albuphine; allylprodine; alphacetylmethadol; benzethidine; benzylfentanyl; benzylmorphine; betacetylmethadol; betameprodine; betamethadol; beta-methyl fentanyl; betaprodine; buprenorphine; butorphanol; butyryl fentanyl; carfentanil; clonitazene; cocaine; codeine; codeine methylbromide; codeine-N-oxide; cyclopropyl fentanyl; cyprenorphine; desomorphine; dextromoramide; dextrorphan; diampromide; diethylthiambutene; difenoxin; dihydromorphine; dimenoxadol; dimepheptanol; dimethylthiambutene; dioxaphetyl butyrate; dipipanone; drotebanol; ethylmethylthiambutene; etonitazene; etorphine; etoxeridine; fentanyl carbamate; 2′-fluoro ortho-fluorofentanyl; 4-fluoroisobutyryl fentanyl; furanyl fentanyl; furethidine; heroin; hydrocodone; hydromorphinol; hydromorphone; β-hydroxy-3-methylfentanyl; β-hydroxyfentanyl; hydroxypethidine; β-hydroxythiofentanyl; ketobemidone; levomoramide; levophenacylmorphan; levorphanol; meperidine; methadone; methoxyacetyl fentanyl; methyldesorphine; methyldihydromorphine; α-meprodine; α-methadol; α-methylfentanyl; α-methylthiofentanyl; 1-methyl-4-phenyl-4-propionoxypiperidine; 3-methylfentanyl; 3-methylthiofentanyl; 4′-methyl acetyl fentanyl; morpheridine; morphine; morphine methylbromide; morphine methylsulfonate; morphine-N-oxide; MT-45; myrophine; nicocodeine; nicomorphine; noracymethadol; norlevorphanol; normethadone; normorphine; norpipanone; ocfentanil; ortho-fluoroacryl fentanyl; ortho-fluorobutyryl fentanyl; ortho-fluorofentanyl; ortho-fluoroisobutyryl fentanyl; ortho-methyl acetylfentanyl; ortho-methyl methoxyacetyl; oxycodone; oxymorphone; para-fluorobutyryl fentanyl; para-fluorofentanyl; para-methylfentanyl (4-methylfentanyl); pentazocine; phenadoxone; phenampromide; phenomorphan; phenoperidine; phenyl fentanyl (benzoyl fentanyl); β-phenyl fentanyl; 1-(-2-phenethyl)-4-phenyl-4-acetoxypiperidine (PEPAP); phoclodine; piritramide; proheptazine; properidine; propiram; propoxyphene; racemoramide; remifentanil; sufentanil; thebacon; tapentadol; thenylfentanyl; thiofentanyl; thiofuranyl fentanyl; tilidine; tramadol; trimeperidine; U-47700; or combinations thereof. In another aspect, the polymer is a polymer that stabilizes dispersion of the GNPs in a solution. In another aspect, the polymer-ligand conjugate comprises:

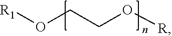

wherein R₁ is the ligand, R is H or C₁₋₄alkyl or hydroxyalkyl, and n is 10 to 11000. In another aspect, the ligand is selected from acetorphine; acetyl fentanyl; acetyldihydrocodeine; acetylmethadol; acetyl-α-methylfentanyl; acryl fentanyl; AH-7921; albuphine; allylprodine; alphacetylmethadol; benzethidine; benzylfentanyl; benzylmorphine; betacetylmethadol; betameprodine; betamethadol; beta-methyl fentanyl; betaprodine; buprenorphine; butorphanol; butyryl fentanyl; carfentanil; clonitazene; cocaine; codeine; codeine methylbromide; codeine-N-oxide; cyclopropyl fentanyl; cyprenorphine; desomorphine; dextromoramide; dextrorphan; diampromide; diethylthiambutene; difenoxin; dihydromorphine; dimenoxadol; dimepheptanol; dimethylthiambutene; dioxaphetyl butyrate; dipipanone; drotebanol; ethylmethylthiambutene; etonitazene; etorphine; etoxeridine; fentanyl carbamate; 2′-fluoro ortho-fluorofentanyl; 4-fluoroisobutyryl fentanyl; furanyl fentanyl; furethidine; heroin; hydrocodone; hydromorphinol; hydromorphone; β-hydroxy-3-methylfentanyl; β-hydroxyfentanyl; hydroxypethidine; β-hydroxythiofentanyl; ketobemidone; levomoramide; levophenacylmorphan; levorphanol; meperidine; methadone; methoxyacetyl fentanyl; methyldesorphine; methyldihydromorphine; α-meprodine; α-methadol; α-methylfentanyl; α-methylthiofentanyl; 1-methyl-4-phenyl-4-propionoxypiperidine; 3-methylfentanyl; 3-methylthiofentanyl; 4′-methyl acetyl fentanyl; morpheridine; morpheridine; morphine; morphine methylbromide; morphine methylsulfonate; morphine-N-oxide; MT-45; myrophine; nalmefene; naloxone; naltrexone; methylnaltrexone; nicocodeine; nicomorphine; noracymethadol; norlevorphanol; normethadone; normorphine; norpipanone; ocfentanil; ortho-fluoroacryl fentanyl; ortho-fluorobutyryl fentanyl; ortho-fluorofentanyl; ortho-fluoroisobutyryl fentanyl; ortho-methyl acetylfentanyl; ortho-methyl methoxyacetyl; oxycodone; oxymorphone; para-fluorobutyryl fentanyl; para-fluorofentanyl; para-methylfentanyl (4-methylfentanyl); pentazocine; phenadoxone; phenampromide; phenomorphan; phenoperidine; phenyl fentanyl (benzoyl fentanyl); β-phenyl fentanyl; 1-(-2-phenethyl)-4-phenyl-4-acetoxypiperidine (PEPAP); phoclodine; piritramide; proheptazine; properidine; propiram; propoxyphene; racemoramide; remifentanil; sufentanil; thebacon; tapentadol; thenylfentanyl; thiofentanyl; thiofuranyl fentanyl; tilidine; tramadol; trimeperidine; U-47700; or combinations thereof. In another aspect, the ligand is naloxone. In another aspect, the polymer-ligand conjugate comprises:

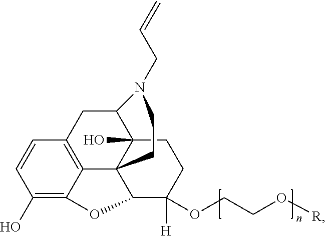

wherein R is H or C₁₋₄alkyl or hydroxyalkyl, and n is 10 to 11000. In another aspect, the system further comprises one or more salts, buffers, organic additives, or a combination thereof. In another aspect, the one or more salts comprise sodium chloride, potassium chloride, sodium phosphate, potassium phosphate, sodium bromide, sodium fluoride, or combinations thereof. In another aspect, the one or more organic additives comprise one or more of benzyl mercaptan, urea, thiourea, cysteamine hydrochloride, tris(hydroxymethyl)-aminomethane (Tris), ethanol amine (EtA), glutathione (GSH), or poly(N-isopropylacrylamide) (PNIPAM). In another aspect, the one or more salts are present at a concentration of about 0.05 M to about 2.0 M. In another aspect, the one or more salts are present at a concentration of about 0.1 M to about 0.3 M. In another aspect, a ratio of GNPs:binding receptor:conjugate is about 1:5:30 to about 1:1000:4000. In another aspect, the system further comprises one or more color masking agents, preservatives, surfactants, viscosifying agents, rheological modifying agents, thickening agents, anti-sagging agents, anti-freezing agents, solvents, or combinations thereof. In another aspect, the system is a solid or a liquid. In another aspect, the system is stable at room temperature for at least about 4 months as a solid.

Another embodiment described herein is a system for colorimetric detection of an opioid, the system comprising: plasmonic gold nanoparticles (GNPs); a water-soluble mu-opioid receptor (wsMOR) conjugated or adsorbed to a surface of the GNPs; a polymer-ligand conjugate comprising polyethylene glycol (PEG) conjugated to naloxone; and about 0.05 M to about 2.0 M of one or more salts.

Another embodiment described herein is a method for colorimetric detection of a target analyte, the method comprising: contacting one or more surfaces or solutions with a system comprising: plasmonic gold nanoparticles (GNPs); a binding receptor conjugated or adsorbed to a surface of the GNPs; and a polymer-ligand conjugate; and detecting emergence of a color wavelength or detecting a color change on the one or more surfaces or solutions, indicating existence of the target analyte. In one aspect, contacting the one or more surfaces or solutions with the system comprises spraying the system onto the one or more surfaces or adding the system to the solution. In another aspect, detecting the color change on the one or more surfaces or solutions comprises detecting a red-to-blue color change when the target analyte is indicated. In another aspect, detecting emergence of the color wavelength on the one or more surfaces or solutions comprises detecting emergence of a blue color wavelength in a range of 600-750 nm when the target analyte is indicated. In another aspect, detecting the color change on the one or more surfaces or solutions comprises detecting an absorbance at wavelengths of 600-750 nm and 520-540 nm and calculating a ratio of the absorbances (A₆₀₀₋₇₅₀/A₅₂₀₋₅₄₀) that is ≥0.2 when the target analyte is indicated. In another aspect, detecting the color change on the one or more surfaces or solutions comprises detecting an absorbance at wavelengths of 650 nm and 524 nm and calculating an absorbance ratio of (A₆₅₀/A₅₂₄) that is ≥0.2 when the target analyte is indicated. In another aspect, the ratio of the absorbances (A₆₀₀₋₇₅₀/A₅₂₀₋₅₄₀) is ≥0.8 when the target analyte is indicated. In another aspect, the method has a limit of detection for the target analyte of about 0.5 μg/mL to about 5 μg/mL. In another aspect, the method has a limit of detection for the target analyte of less than about 0.5 μg/mL. In another aspect, the method is performed in under 10 minutes.

Another embodiment described herein is a method for colorimetric detection of an opioid, the method comprising: contacting one or more surfaces or solutions with a system comprising: plasmonic gold nanoparticles (GNPs); an opioid-binding receptor conjugated or adsorbed to a surface of the GNPs; and a polymer-ligand conjugate comprising a polymer conjugated to an opioid ligand; and detecting emergence of a blue color wavelength in a range of 600-750 nm or detecting a red-to-blue color change on the one or more surfaces or solutions, indicating existence of the opioid. In one aspect, the opioid-binding receptor is a water-soluble mu-opioid receptor (wsMOR). In another aspect, the opioid is one or more of acetorphine; acetyl fentanyl; acetyldihydrocodeine; acetylmethadol; acetyl-α-methylfentanyl; acryl fentanyl; AH-7921; albuphine; allylprodine; alphacetylmethadol; benzethidine; benzylfentanyl; benzylmorphine; betacetylmethadol; betameprodine; betamethadol; beta-methyl fentanyl; betaprodine; buprenorphine; butorphanol; butyryl fentanyl; carfentanil; clonitazene; cocaine; codeine; codeine methylbromide; codeine-N-oxide; cyclopropyl fentanyl; cyprenorphine; desomorphine; dextromoramide; dextrorphan; diampromide; diethylthiambutene; difenoxin; dihydromorphine; dimenoxadol; dimepheptanol; dimethylthiambutene; dioxaphetyl butyrate; dipipanone; drotebanol; ethylmethylthiambutene; etonitazene; etorphine; etoxeridine; fentanyl carbamate; 2′-fluoro ortho-fluorofentanyl; 4-fluoroisobutyryl fentanyl; furanyl fentanyl; furethidine; heroin; hydrocodone; hydromorphinol; hydromorphone; β-hydroxy-3-methylfentanyl; β-hydroxyfentanyl; hydroxypethidine; β-hydroxythiofentanyl; ketobemidone; levomoramide; levophenacylmorphan; levorphanol; meperidine; methadone; methoxyacetyl fentanyl; methyldesorphine; methyldihydromorphine; α-meprodine; α-methadol; α-methylfentanyl; α-methylthiofentanyl; 1-methyl-4-phenyl-4-propionoxypiperidine; 3-methylfentanyl; 3-methylthiofentanyl; 4′-methyl acetyl fentanyl; morpheridine; morphine; morphine methylbromide; morphine methylsulfonate; morphine-N-oxide; MT-45; myrophine; nicocodeine; nicomorphine; noracymethadol; norlevorphanol; normethadone; normorphine; norpipanone; ocfentanil; ortho-fluoroacryl fentanyl; ortho-fluorobutyryl fentanyl; ortho-fluorofentanyl; ortho-fluoroisobutyryl fentanyl; ortho-methyl acetylfentanyl; ortho-methyl methoxyacetyl; oxycodone; oxymorphone; para-fluorobutyryl fentanyl; para-fluorofentanyl; para-methylfentanyl (4-methylfentanyl); pentazocine; phenadoxone; phenampromide; phenomorphan; phenoperidine; phenyl fentanyl (benzoyl fentanyl); β-phenyl fentanyl; 1-(-2-phenethyl)-4-phenyl-4-acetoxypiperidine (PEPAP); phoclodine; piritramide; proheptazine; properidine; propiram; propoxyphene; racemoramide; remifentanil; sufentanil; thebacon; tapentadol; thenylfentanyl; thiofentanyl; thiofuranyl fentanyl; tilidine; tramadol; trimeperidine; or U-47700. In another aspect, the polymer is polyethylene glycol (PEG) having a molecular weight >1000 Da and the opioid ligand is naloxone. In another aspect, the polymer-ligand conjugate is:

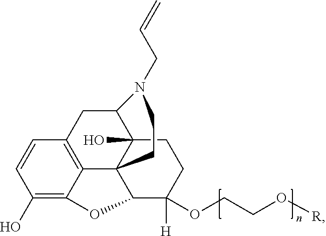

wherein R is H or C₁₋₄alkyl or hydroxyalkyl, and n is 10 to 11000. In another aspect, the system further comprises one or more salts selected from sodium chloride, potassium chloride, sodium phosphate, potassium phosphate, sodium bromide, sodium fluoride, or combinations thereof.

Another embodiment described herein is the use of a colorimetric detection system as a reagent, research tool, or forensics tool for colorimetric detection of a target analyte, the system comprising: plasmonic gold nanoparticles (GNPs); a binding receptor conjugated or adsorbed to a surface of the GNPs; a polymer-ligand conjugate; and one or more salts.

Another embodiment described herein is a kit for colorimetric detection of a target analyte, the kit comprising: a system comprising: plasmonic gold nanoparticles (GNPs); a binding receptor conjugated or adsorbed to a surface of the GNPs; a polymer-ligand conjugate; and one or more salts; a system applicator; optionally, a detection device; optionally, one or more articles of personal protective equipment; optionally, one or more buffers or receptacles; and optionally, one or more of packaging or instructions for use.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic illustration of opioid detection based on the switch of well dispersed GNPs to aggregated GNPs, which is caused by competitive replacement of the Naloxone-PEG conjugate (i.e., Naloxol-PEG) from the wsMOR by an opioid target analyte. wsMOR: water soluble mu-opioid receptor. PEG: Polyethylene glycol polymer.

FIG. 2A-C show colorimetric detection of fentanyl with different concentrations by using AuNP/MOR/Naloxol-PEG10K solution. Sample formulation: AuNP:MOR:Naloxol-PEG=1:20:400 in 0.30 M NaCl solution. FIG. 2A shows photographs of the color response of the detection solution to fentanyl with different concentrations. FIG. 2B shows the UV/Vis spectra for various concentrations of fentanyl. FIG. 2C shows a graph illustrating the A₆₅₀/A₅₂₄ absorbance ratio for each concentration. Spectra were measured at 10 min after addition of fentanyl.

FIG. 3A-B show colorimetric detection of fentanyl derivatives at different concentrations by a detection formulation: AuNP:MOR:Naloxol-PEG=1:20:400 in 0.3 M NaCl solution. FIG. 3A shows photographs of samples of sufentanil, carfentanil, acetylfentanyl, and remifentanil being detected by the detection formulation. The samples in black frames have A₆₅₀/A₅₂₄ ratios greater than 0.8. FIG. 3B shows the A₆₅₀/A₅₂₄ ratio as a function of the analyte concentration. The dashed line represents the A₆₅₀/A₅₂₄ ratio of 0.8.

FIG. 4A-B show colorimetric response of the detection solution exposed to potential interferents with or without fentanyl. The photographs were taken at 10 min after adding interferent compounds and fentanyl. FIG. 4B shows the A₆₅₀/A₅₂₄ absorbance ratio with (black bars) or without fentanyl (open bars). The concentration of interferent and fentanyl are 500 mg/L and 10 mg/L, respectively.

FIG. 5A-C show the effects of interferent compounds on fentanyl detection. FIG. 5A shows photographs of the color response of the detection solution at 10 min after adding interferent compounds and a mixture of interferent compounds and fentanyl. FIG. 5B shows the A₆₅₀/A₅₂₄ absorbance ratio of the detection solution in presence of different interferent compound mixtures. FIG. 5C shows the UV/Vis spectrum of the testing solution with inorganic solid suspension interferents with (Sample C1) and without fentanyl (Sample C2). Sample details: A1: blank detection solution, A2: blank detection solution+fentanyl; B1: detection solution+sugar mixture, B2: detection solution+sugar mixture+fentanyl, C1: detection solution+inorganic solid particle mixture, C2: detection solution+inorganic solid particle mixture+fentanyl. D1: detection solution+inorganic metal salts mixture, D2: detection solution+inorganic metal salts mixture+fentanyl. The sugar mixture is comprised of 500 mg/L of each of dextrose, fructose, lactose, and sucrose for a total of 2000 mg/L. The inorganic solid particle mixture is comprised of 500 mg/L of each of Al₂O₃, CaCO₃, SiO₂, and sand fine particles. The inorganic metal salts mixture is comprised of 500 mg/L of each of CaCl₂), MgSO₄, NaH₂PO₄, and ZnSO₄.

FIG. 6A-B show colorimetric response of AuNPs/SAV/Biotin-PEG (5K) to biotin target and different interferent samples. FIG. 6A shows photographs of the colorimetric response of detection solution to biotin and various inorganic interferents. FIG. 6B shows the UV/Vis spectra of the detection solution after adding biotin and inorganic interferents. Sample molar ratio: AuNPs:SAV:Biotin-PEG (5K)=1:20:600, in 0.1 M NaCl solution.

### DETAILED DESCRIPTION

Unless otherwise defined, all technical and scientific terms used herein have the same meaning as commonly understood by one of ordinary skill in the art. For example, any nomenclatures used in connection with, and techniques of chemistry, biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjuctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points.

As used herein, the terms “room temperature,” “RT,” or “ambient temperature” refer to the typical temperature in an indoor laboratory setting. In one aspect, the laboratory setting is climate controlled to maintain the temperature at a substantially uniform temperature or with a specific range of temperatures. In one aspect, “room temperature” refers a temperature of about 20-30° C., including all integers and endpoints within the specified range. In another aspect, “room temperature” refers a temperature of about 20-30° C.; about 22-30° C.; about 25-30° C.; about 27-30° C.; about 20-22° C.; about 20-25° C.; about 20-27° C.; about 22-25° C.; about 22-27° C.; about 25-27° C.; about 20° C.±10%; about 22° C.±10%; about 25° C.±10%; about 27° C.±10%; ˜20° C., ˜22° C., ˜25° C., or ˜27° C., at standard atmospheric pressure.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, the term “opioid” refers to a general compound designation for all exogenous substances that bind stereo-specifically to any of several subspecies of opioid receptors and produce agonist actions. Opioids are a group of drugs that exhibit opium- or morphine-like properties and effects. The opioids are employed primarily as moderate to strong analgesics, but have many other pharmacological effects as well, including drowsiness, respiratory depression, and changes in mood and mental clouding, without a resulting loss of consciousness. For purposes of the present invention, the term “opioid” shall include an opioid and its pharmaceutically acceptable salts thereof; stereoisomers thereof, ethers and esters thereof; or mixtures thereof. In certain non-limiting exemplary embodiments of the present invention, a target analyte may include an opioid selected from acetorphine; acetyl fentanyl; acetyldihydrocodeine; acetylmethadol; acetyl-α-methylfentanyl; acryl fentanyl; AH-7921; albuphine; allylprodine; alphacetylmethadol; benzethidine; benzylfentanyl; benzylmorphine; betacetylmethadol; betameprodine; betamethadol; beta-methyl fentanyl; betaprodine; buprenorphine; butorphanol; butyryl fentanyl; carfentanil; clonitazene; cocaine; codeine; codeine methylbromide; codeine-N-oxide; cyclopropyl fentanyl; cyprenorphine; desomorphine; dextromoramide; dextrorphan; diampromide; diethylthiambutene; difenoxin; dihydromorphine; dimenoxadol; dimepheptanol; dimethylthiambutene; dioxaphetyl butyrate; dipipanone; drotebanol; ethylmethylthiambutene; etonitazene; etorphine; etoxeridine; fentanyl carbamate; 2′-fluoro ortho-fluorofentanyl; 4-fluoroisobutyryl fentanyl; furanyl fentanyl; furethidine; heroin; hydrocodone; hydromorphinol; hydromorphone; β-hydroxy-3-methylfentanyl; β-hydroxyfentanyl; hydroxypethidine; β-hydroxythiofentanyl; ketobemidone; levomoramide; levophenacylmorphan; levorphanol; meperidine; methadone; methoxyacetyl fentanyl; methyldesorphine; methyldihydromorphine; α-meprodine; α-methadol; α-methylfentanyl; α-methylthiofentanyl; 1-methyl-4-phenyl-4-propionoxypiperidine; 3-methylfentanyl; 3-methylthiofentanyl; 4′-methyl acetyl fentanyl; morpheridine; morphine; morphine methylbromide; morphine methylsulfonate; morphine-N-oxide; MT-45; myrophine; nicocodeine; nicomorphine; noracymethadol; norlevorphanol; normethadone; normorphine; norpipanone; ocfentanil; ortho-fluoroacryl fentanyl; ortho-fluorobutyryl fentanyl; ortho-fluorofentanyl; ortho-fluoroisobutyryl fentanyl; ortho-methyl acetylfentanyl; ortho-methyl methoxyacetyl; oxycodone; oxymorphone; para-fluorobutyryl fentanyl; para-fluorofentanyl; para-methylfentanyl (4-methylfentanyl); pentazocine; phenadoxone; phenampromide; phenomorphan; phenoperidine; phenyl fentanyl (benzoyl fentanyl); β-phenyl fentanyl; 1-(-2-phenethyl)-4-phenyl-4-acetoxypiperidine (PEPAP); phoclodine; piritramide; proheptazine; properidine; propiram; propoxyphene; racemoramide; remifentanil; sufentanil; thebacon; tapentadol; thenylfentanyl; thiofentanyl; thiofuranyl fentanyl; tilidine; tramadol; trimeperidine; U-47700; or combinations thereof. In some embodiments, the opioid is one or more of fentanyl, sufentantil, carfentanil, remifentanil, or acetylfentanyl.

As used herein, the term “ligand” refers to a molecular species or compound having binding affinity to a binding receptor of the disclosed compositions and methods, where the ligand non-covalently binds to the binding receptor. The “binding receptor” refers to any biomolecules that can selectively interact with the ligand or a target analyte through non-covalent interactions. As used herein, an “opioid ligand” refers to any molecule that binds to an opioid-binding receptor, including, but not limited to, opioids and synthetic opioid receptor antagonists. In some embodiments, the binding receptor is an opioid-binding receptor and the ligand is an opioid ligand. In some embodiments, the ligand is chemically attached or conjugated to a polymer moiety to generate a polymer-ligand conjugate. In some embodiments, the ligand of a polymer-ligand conjugate is the same molecule as a target analyte. In other embodiments, the ligand of a polymer-ligand conjugate is a different molecule from a target analyte. When attached to a nanoparticle surface and bound to a receptor, the polymer-ligand conjugate may work as a molecular spacer to maintain proper distance between nanoparticles and proper dispersion of the nanoparticles. In the presence of specific target analytes (e.g., opioids), the polymer-ligand conjugates are selectively and competitively replaced and the aggregation of the plasmonic GNPs is triggered, resulting in clear colorimetric responses due to the plasmonic interparticle coupling effect, which can be directly observed by the naked eye and/or a UV-vis spectrophotometer. In certain non-limiting exemplary embodiments of the present invention, a ligand may be selected from acetorphine; acetyl fentanyl; acetyldihydrocodeine; acetylmethadol; acetyl-α-methylfentanyl; acryl fentanyl; AH-7921; albuphine; allylprodine; alphacetylmethadol; benzethidine; benzylfentanyl; benzylmorphine; betacetylmethadol; betameprodine; betamethadol; beta-methyl fentanyl; betaprodine; buprenorphine; butorphanol; butyryl fentanyl; carfentanil; clonitazene; cocaine; codeine; codeine methylbromide; codeine-N-oxide; cyclopropyl fentanyl; cyprenorphine; desomorphine; dextromoramide; dextrorphan; diampromide; diethylthiambutene; difenoxin; dihydromorphine; dimenoxadol; dimepheptanol; dimethylthiambutene; dioxaphetyl butyrate; dipipanone; drotebanol; ethylmethylthiambutene; etonitazene; etorphine; etoxeridine; fentanyl carbamate; 2′-fluoro ortho-fluorofentanyl; 4-fluoroisobutyryl fentanyl; furanyl fentanyl; furethidine; heroin; hydrocodone; hydromorphinol; hydromorphone; β-hydroxy-3-methylfentanyl; β-hydroxyfentanyl; hydroxypethidine; β-hydroxythiofentanyl; ketobemidone; levomoramide; levophenacylmorphan; levorphanol; meperidine; methadone; methoxyacetyl fentanyl; methyldesorphine; methyldihydromorphine; α-meprodine; α-methadol; α-methylfentanyl; α-methylthiofentanyl; 1-methyl-4-phenyl-4-propionoxypiperidine; 3-methylfentanyl; 3-methylthiofentanyl; 4′-methyl acetyl fentanyl; morpheridine; morpheridine; morphine; morphine methylbromide; morphine methylsulfonate; morphine-N-oxide; MT-45; myrophine; nalmefene; naloxol; naloxone; naltrexone; methylnaltrexone; nicocodeine; nicomorphine; noracymethadol; norlevorphanol; normethadone; normorphine; norpipanone; ocfentanil; ortho-fluoroacryl fentanyl; ortho-fluorobutyryl fentanyl; ortho-fluorofentanyl; ortho-fluoroisobutyryl fentanyl; ortho-methyl acetylfentanyl; ortho-methyl methoxyacetyl; oxycodone; oxymorphone; para-fluorobutyryl fentanyl; para-fluorofentanyl; para-methylfentanyl (4-methylfentanyl); pentazocine; phenadoxone; phenampromide; phenomorphan; phenoperidine; phenyl fentanyl (benzoyl fentanyl); β-phenyl fentanyl; 1-(-2-phenethyl)-4-phenyl-4-acetoxypiperidine (PEPAP); phoclodine; piritramide; proheptazine; properidine; propiram; propoxyphene; racemoramide; remifentanil; sufentanil; thebacon; tapentadol; thenylfentanyl; thiofentanyl; thiofuranyl fentanyl; tilidine; tramadol; trimeperidine; U-47700; or combinations thereof. In some embodiments, the ligand is naloxone.

As used herein, the term “C₁₋₄alkyl” means a straight or branched chain hydrocarbon containing from 1 to 4 carbon atoms.

As used herein, the term “hydroxyalkyl,” as used herein, means at least one —OH group, is appended to the parent molecular moiety through an alkylene group, as defined herein.

As used herein, the terms “limit of detection” or “LOD” refer to the lowest signal, or the lowest corresponding quantity to be determined from the signal, that can be measured or observed with a sufficient degree of confidence or statistical significance. In some embodiments of the present invention, disclosed methods have a limit of detection for a target analyte of about 0.5 μg/mL to about 5 μg/mL. In other embodiments, disclosed methods have a limit of detection for a target analyte of less than about 0.5 μg/mL.

As used herein, the terms “conjugated” or “conjugate” refer to a covalent chemical attachment between two molecular species. In some embodiments of the present invention, disclosed compositions may include a binding receptor that is either conjugated or physically adsorbed to a surface of GNPs.

As used herein, the term “detecting” may refer to a means of acquiring information (e.g., color change) via one or more sensing elements. Detecting may include visual observation (i.e., using the “naked eye”) or actions of measuring, quantifying, qualifying, estimating, sensing, calculating, interpolating, extrapolating, inferring, deducing, or any combination of these actions. Detecting may include the use of the “naked eye,” but also may include the use of digital or computational systems or devices for detection.

Currently, many different techniques exist for detection of opioid compounds and derivatives from different types of samples. The most common techniques, which are routinely in use, are high performance liquid chromatography (HPLC), absorption spectroscopy, infrared (IR) spectroscopy, thin layer chromatography, mass spectrometry (MS), and gas chromatography (GC). However, these methods require sophisticated equipment, which are quite inconvenient for outdoor detection or for on-spot analysis.

Colorimetric methods have been developed to detect opioid compound, including dye chemistry and unmodified gold nanoparticles (GNPs). See e.g., WO 2015006720 A1; Kammer, et al., Anal. Chem. 91, 10582-10588 (2019); and Lodha, et al., RSC Adv. 4: 50443-50448 (2014). In the dye chemistry approach, colored compound is generated through multiple step reactions and usually the toxic reagents are used in the detection process. The detection process is complicated, and the sensitivity is low. The mechanism of the unmodified citrate capped GNPs approach is that the opioids possess many hydrogen bond donor/acceptor sites, including hydroxyl, methoxy, and furan-like oxygen hybrid rings, which provide the link between citrate and opioids to induce the aggregations of GNPs. This approach lacks good selectivity and sensitivity, since the aggregation of GNPs can be induced by many interferents.

Described herein is a universal colorimetric method for biomolecule and small molecule detection. In some embodiments, the detection mechanism and system are based on the use of unique plasmonic nanoparticles that are protected and stabilized by the complexes of the biomolecular “receptors” and its PEGylated ligands, where the “receptors” are adsorbed onto surfaces of the plasmonic nanoparticles (NPs) and the PEGylated ligands work as a spacer to maintain proper distance between the plasmonic NPs and to keep the plasmonic NPs in a well-dispersed state and display a size specific color. In the presence of specific target analytes, the pegylated ligands are selectively and competitively replaced by the target analyte. As the pegylated ligands are replaced, the area on the GNPs' surface may be exposed to a salt in the solution such as NaCl, and the aggregation of the plasmonic GNPs is triggered, resulting in clear colorimetric responses due to the plasmonic interparticle coupling effect which can be directly observed by naked eye and UV-vis spectrophotometer. The replacement happens in all receptor-ligand pairs to their thermodynamic equilibrium. Importantly, when the ligand is pegylated, the equilibrium dissociation constants of pegylated ligand with receptor will increase compared with that of the original ligand-receptor complex. This phenomenon improves the replacement probability as well as the detection sensitivity.

Plasmonic nanoparticles (NPs)—including gold, silver, and platinum particles-possess unique localized surface plasmon resonance (LSPR) properties with high molar extinction coefficients and show size-dependent, distinct color changes from dispersed single particle to aggregated particles. Owing to its simple, cost-effective, and easy to interpretate features, the aggregation-based colorimetric assays have been widely applied in small molecules and biomolecules sensing.

Current aggregation-based plasmonic nanoparticle colorimetric assays can be divided into two systems: label-free and labeled. The label free system is based on unmodified plasmonic NPs whose aggregation and colorimetric responses can be easily triggered in the presence of analytes by neutralizing the surface charges or inducing cross-linking of the plasmonic NPs. Despite its simplicity, the major disadvantage of this label free system is the lack of selectivity and specificity to the target analyte due to its poor stability. On the other hand, in the labeled system, also called the crosslinking system, the plasmonic NPs surfaces are modified by biomolecules such as DNA, aptamers, peptides or antibodies through chemical linkages prior to detection. The modified plasmonic NPs have higher steric and hydration-based interparticle repulsions and are more stable than unmodified plasmonic NPs. As a result, the specificity and selectivity of the labeled detection system are improved since the aggregation of plasmonic NPs are more specifically and selectively triggered by the target analytes. However, the application of this labeled detection system is limited by three factors. First, there is no versatile approaches to stabilize the biomolecule-functionalized plasmonic NPs as single dispersed particle—direct adsorption of the biomolecule onto the plasmonic NP surfaces, even at a high concentration that allows a monolayer adsorption, is not sufficient to protect the particles from aggregation. Therefore, special linkers/spacers are usually employed to conjugate the biomolecules onto the plasmonic surfaces and to provide proper inter-particle distance to prevent aggregation. Nevertheless, this prolonged inter-particle distance may greatly affect the sensitivity of detection system because the color change effect is size and distance dependent. Second, in this labeled system, strong binding affinity between the biomolecule and the analyte is required to trigger the crosslinking and aggregation of the plasmonic NPs and the consequential colorimetric responses. Third, in this labeled system, the analyte must be able to bind to at least two biomolecular receptors that are attached to at least two different GNPs in order to achieve crosslinking of two or more GNPs.

The methods described herein have been applied to develop a highly specific and sensitive colorimetric assay for the detection of fentanyl and its derivatives. Such colorimetric assay is rapid (i.e., less than 10 minutes), robust and selective in harsh detection environment with high concentrations (2 g/L) of potential interferents such as inorganic salts, solid suspensions, and organic compounds (e.g., sugars). The limit of detection (LOD) of this assay may be less than 1 μg/mL, which is 10-fold more sensitive than or comparable to the literature-reported colorimetric assays for opioid compounds based on chemical dye method and unmodified GNPs.

The aggregation-based colorimetric responses of plasmonic NPs have been widely used for the detections of small molecules and biomolecules, however, a universal platform has yet been established and the aggregation responses can be induced by many interferents and cause false positive results. The method described herein provides a universal platform for the detection of a broad spectrum of small molecules and biomolecules with high selectivity and sensitivity owing to the following attributes. First, the unique plasmonic NPs/receptor/PEGylated ligands system is extremely stable and highly tolerable to general aggregation inducers. Second, based on the highly sensitive and selective biological receptor-ligand binding events, this detection system is able to avoid false positive results in the presence of non-ligand interferents while maintaining a high sensitivity to the analytes. Finally, this is a versatile platform that can be easily applied for the detection of different analytes by simply changing the receptor and PEGylated ligand pair. Other advantages of the method include detection of analyte with naked eye, and no need for advanced and expensive equipment. This technology can be further extended and incorporated to other detection methods, such as point-of-care lateral flow test strips, drug detection kits, and surface mapping detection.

Compared to the popular cross-linking approaches, the strategy overcomes many problems existed in the tradition colorimetric methods:

The concept described herein established a platform to detect broad range of medical and biological compounds, and greatly extended the applicability of the colorimetric detection approaches. In many cases, there is no ideal approaches to stabilize the ligand-functionalized GNPs with proper sensing concentration in the detection environment. For example, a number of ligands and receptors have been tested to functionalize GNPs, such as streptavidin, opioid receptor protein, and other proteins. Even forming a monolayer of these ligands on the GNP surface, the modified GNPs still cannot achieve the desired stability in regular buffer solutions (e.g., carbonate buffer, phosphate buffer, PBS) and NaCl for sufficiently long periods of time to achieve practical applications. In certain cases, the affinity between the analyte and ligands are not strong, which cannot generate the effective crosslinking of the GNPs. In other cases, the interparticle distance of the GNPS in the crosslinked configurations is not shorter enough, and the color change cannot be clearly distinguished. This greatly limit the cross-linking-based detection. The approach described herein successfully overcomes these problems.

The method described herein offers excellent selectivity and sensitivity to the analyte targets due to the specific competitive binding replacement process. Only the analyte targets replace the PEGylated ligand, resulting in high selectivity. The replacement happened in all the receptor-ligand pairs to their thermodynamic equilibrium. In addition, when the target ligand is pegylated, the equilibrium dissociation constants of PEGylated ligand with receptor will increase compared with that of the original ligand-receptor complex. Once part of the pegylated ligands is replaced, the area on the gold nanoparticle surface is exposed to NaCl, and the aggregation of the plasmonic NPs is triggered, resulting in highly sensitive colorimetric responses due to the size-dependent plasmonic coupling effect.

The detection system described herein exhibits high stability. This method is based on the destabilization process and the PEGylated ligand spacer effectively maintain the interparticle distance, avoiding the aggregation of GNPs. Many practical detection systems require that analyte targets are in blood, urine, and polluted water samples and these results can be highly affected by interferents. The detection system described herein has great tolerance to many interferent compounds (e.g., inorganic particles, inorganic salts, and organic compounds) and harsh detection environments. In addition, the detection solution system has a long shelf life (i.e., a minimum of 4 months). The formulation can also be prepared in dry powder formation with a much longer shelf life.

The disclosed colorimetric detection systems are also non-toxic. Unlike the traditional chemistry colorimetric detection approach, in which toxic reagents or corrosive acids or bases are popularly used to generate the coloring compound, the disclosed detection system is comprised of non-toxic compounds, providing high safety during the detection process.

Many colorimetric sensing strategies have been developed to detect biological targets including protein, enzyme, amino acid, nucleic acid, drug compounds. Most of the detection process are based on the crosslinking approach, in which, GNP aggregation is induced by the assembly of ligand-functionalized GNPs with the formation of intermolecular bonds, which overpower the interparticle repulsive forces.

The conventional methodologies lack broad applicability, because in these methods, the detection system needs to be specially designed to each target analyte molecule. The destabilization method is rarely used in the detection process since it required a special reaction mechanism which can cleave part of the ligand and reduce the stability of the GNPs.

The traditional methods are not applicable to many analytes due to many reasons. In some cases, there is no good strategy to stabilize the ligand-functionalized GNPs with proper concentration in the detection environment. In certain cases, the affinity between the analyte and ligands are not strong, which cannot generate the effective crosslinking of the GNPs. In some cases, the interparticle distance of the GNPS in the crosslinked configurations is not shorter enough, and the color change cannot be clearly distinguished. In some cases, the target analyte could not bind to two or more receptors simultaneously, diminishing the possibility of GNP crosslinking.

The methodology described herein is an innovative approach which overcomes the problems in current colorimetric detection approaches. This method has not been described in any previous articles or patents. The innovation is the replacement of covalently PEGylated ligand molecules that bind to the receptor by the target molecules, in which the PEGylated ligand molecules serve as a spacer to maintain the proper distance between the GNPs, thereby avoiding their aggregation. In some embodiments, the ligand of the disclosed PEGylated ligand may even be the target analyte molecule itself. The replacement happens in all the receptor-ligand pairs to their thermodynamic equilibrium. In addition, the equilibrium dissociation constants of PEGylated ligand with receptor greatly increase compared with that of the original ligand-receptor complex. The specific replacement of the PEGylated ligand by the target analyte on the receptor reduces the stability of the GNPs in the detection system, resulting the aggregation of the plasmonic GNPs due to the plasmonic interparticle coupling effect. This offers the detection system high sensitivity and selectivity.

Because the gold surface chemistry and PEGylation chemistry are well developed, the detection system can be designed to utilize various target molecules having affinity to their respective binding receptors. The disclosed methods greatly extend the applicability of colorimetric detection approach toward many biomolecules and medical compounds.

One embodiment described herein is a system for colorimetric detection of a target analyte, the system comprising: plasmonic gold nanoparticles (GNPs); a binding receptor conjugated or adsorbed to a surface of the GNPs; and a polymer-ligand conjugate. In one aspect, the binding receptor is an opioid-binding receptor. In another aspect, the opioid-binding receptor is a water-soluble mu-opioid receptor (wsMOR). In another aspect, the target analyte is selected from acetorphine; acetyl fentanyl; acetyldihydrocodeine; acetylmethadol; acetyl-α-methylfentanyl; acryl fentanyl; albuphine; allylprodine; alphacetylmethadol; benzethidine; benzylfentanyl; benzylmorphine; betacetylmethadol; betameprodine; betamethadol; beta-methyl fentanyl; betaprodine; buprenorphine; butorphanol; butyryl fentanyl; carfentanil; clonitazene; cocaine; codeine; codeine methylbromide; codeine-N-oxide; cyclopropyl fentanyl; cyprenorphine; desomorphine; dextromoramide; dextrorphan; diampromide; diethylthiambutene; difenoxin; dihydromorphine; dimenoxadol; dimepheptanol; dimethylthiambutene; dioxaphetyl butyrate; dipipanone; drotebanol; ethylmethylthiambutene; etonitazene; etorphine; etoxeridine; fentanyl carbamate; 2′-fluoro ortho-fluorofentanyl; 4-fluoroisobutyryl fentanyl; furanyl fentanyl; furethidine; heroin; hydrocodone; hydromorphinol; hydromorphone; β-hydroxy-3-methylfentanyl; β-hydroxyfentanyl; hydroxypethidine; β-hydroxythiofentanyl; ketobemidone; levomoramide; levophenacylmorphan; levorphanol; meperidine; methadone; methoxyacetyl fentanyl; methyldesorphine; methyldihydromorphine; α-meprodine; α-methadol; α-methylfentanyl; α-methylthiofentanyl; 1-methyl-4-phenyl-4-propionoxypiperidine; 3-methylfentanyl; 3-methylthiofentanyl; 4′-methyl acetyl fentanyl; morpheridine; morphine; morphine methylbromide; morphine methylsulfonate; morphine-N-oxide; MT-45; myrophine; nicocodeine; nicomorphine; noracymethadol; norlevorphanol; normethadone; normorphine; norpipanone; ocfentanil; ortho-fluoroacryl fentanyl; ortho-fluorobutyryl fentanyl; ortho-fluorofentanyl; ortho-fluoroisobutyryl fentanyl; ortho-methyl acetylfentanyl; ortho-methyl methoxyacetyl; oxycodone; oxymorphone; para-fluorobutyryl fentanyl; para-fluorofentanyl; para-methylfentanyl (4-methylfentanyl); pentazocine; phenadoxone; phenampromide; phenomorphan; phenoperidine; phenyl fentanyl (benzoyl fentanyl); β-phenyl fentanyl; 1-(-2-phenethyl)-4-phenyl-4-acetoxypiperidine (PEPAP); phoclodine; piritramide; proheptazine; properidine; propiram; propoxyphene; racemoramide; remifentanil; sufentanil; thebacon; tapentadol; thenylfentanyl; thiofentanyl; thiofuranyl fentanyl; tilidine; tramadol; trimeperidine; U-47700; or combinations thereof. In another aspect, the polymer is a polymer that stabilizes dispersion of the GNPs in a solution. In another aspect, the polymer-ligand conjugate comprises:

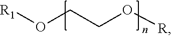

wherein R₁ is the ligand, R is H or C₁₋₄alkyl or hydroxyalkyl, and n is 10 to 11000. In another aspect, the ligand is selected from acetorphine; acetyl fentanyl; acetyldihydrocodeine; acetylmethadol; acetyl-α-methylfentanyl; acryl fentanyl; AH-7921; albuphine; allylprodine; alphacetylmethadol; benzethidine; benzylfentanyl; benzylmorphine; betacetylmethadol; betameprodine; betamethadol; beta-methyl fentanyl; betaprodine; buprenorphine; butorphanol; butyryl fentanyl; carfentanil; clonitazene; cocaine; codeine; codeine methylbromide; codeine-N-oxide; cyclopropyl fentanyl; cyprenorphine; desomorphine; dextromoramide; dextrorphan; diampromide; diethylthiambutene; difenoxin; dihydromorphine; dimenoxadol; dimepheptanol; dimethylthiambutene; dioxaphetyl butyrate; dipipanone; drotebanol; ethylmethylthiambutene; etonitazene; etorphine; etoxeridine; fentanyl carbamate; 2′-fluoro ortho-fluorofentanyl; 4-fluoroisobutyryl fentanyl; furanyl fentanyl; furethidine; heroin; hydrocodone; hydromorphinol; hydromorphone; β-hydroxy-3-methylfentanyl; β-hydroxyfentanyl; hydroxypethidine; β-hydroxythiofentanyl; ketobemidone; levomoramide; levophenacylmorphan; levorphanol; meperidine; methadone; methoxyacetyl fentanyl; methyldesorphine; methyldihydromorphine; α-meprodine; α-methadol; α-methylfentanyl; a-methylthiofentanyl; 1-methyl-4-phenyl-4-propionoxypiperidine; 3-methylfentanyl; 3-methylthiofentanyl; 4′-methyl acetyl fentanyl; morpheridine; morpheridine; morphine; morphine methylbromide; morphine methylsulfonate; morphine-N-oxide; MT-45; myrophine; nalmefene; naloxone; naltrexone; methylnaltrexone; nicocodeine; nicomorphine; noracymethadol; norlevorphanol; normethadone; normorphine; norpipanone; ocfentanil; ortho-fluoroacryl fentanyl; ortho-fluorobutyryl fentanyl; ortho-fluorofentanyl; ortho-fluoroisobutyryl fentanyl; ortho-methyl acetylfentanyl; ortho-methyl methoxyacetyl; oxycodone; oxymorphone; para-fluorobutyryl fentanyl; para-fluorofentanyl; para-methylfentanyl (4-methylfentanyl); pentazocine; phenadoxone; phenampromide; phenomorphan; phenoperidine; phenyl fentanyl (benzoyl fentanyl); β-phenyl fentanyl; 1-(-2-phenethyl)-4-phenyl-4-acetoxypiperidine (PEPAP); phoclodine; piritramide; proheptazine; properidine; propiram; propoxyphene; racemoramide; remifentanil; sufentanil; thebacon; tapentadol; thenylfentanyl; thiofentanyl; thiofuranyl fentanyl; tilidine; tramadol; trimeperidine; U-47700; or combinations thereof. In another aspect, the ligand is naloxone. In another aspect, the polymer-ligand conjugate comprises:

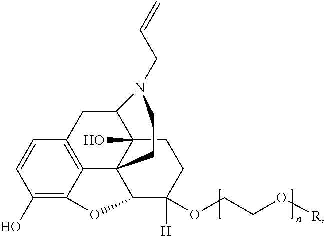

wherein R is H or C₁₋₄alkyl or hydroxyalkyl, and n is 10 to 11000. In another aspect, the system further comprises one or more salts, buffers, organic additives, or a combination thereof. In another aspect, the one or more salts comprise sodium chloride, potassium chloride, sodium phosphate, potassium phosphate, sodium bromide, sodium fluoride, or combinations thereof. In another aspect, the one or more organic additives comprise one or more of benzyl mercaptan, urea, thiourea, cysteamine hydrochloride, tris(hydroxymethyl)-aminomethane (Tris), ethanol amine (EtA), glutathione (GSH), or poly(N-isopropylacrylamide) (PNIPAM). In another aspect, the one or more salts are present at a concentration of about 0.05 M to about 2.0 M. In another aspect, the one or more salts are present at a concentration of about 0.1 M to about 0.3 M. In another aspect, a ratio of GNPs:binding receptor:conjugate is about 1:5:30 to about 1:1000:4000. In another aspect, the system further comprises one or more color masking agents, preservatives, surfactants, viscosifying agents, rheological modifying agents, thickening agents, anti-sagging agents, anti-freezing agents, solvents, or combinations thereof. In another aspect, the system is a solid or a liquid. In another aspect, the system is stable at room temperature for at least about 4 months as a solid.

Another embodiment described herein is a system for colorimetric detection of an opioid, the system comprising: plasmonic gold nanoparticles (GNPs); a water-soluble mu-opioid receptor (wsMOR) conjugated or adsorbed to a surface of the GNPs; a polymer-ligand conjugate comprising polyethylene glycol (PEG) conjugated to naloxone; and about 0.05 M to about 2.0 M of one or more salts.

Another embodiment described herein is a method for colorimetric detection of a target analyte, the method comprising: contacting one or more surfaces or solutions with a system comprising: plasmonic gold nanoparticles (GNPs); a binding receptor conjugated or adsorbed to a surface of the GNPs; and a polymer-ligand conjugate; and detecting emergence of a color wavelength or detecting a color change on the one or more surfaces or solutions, indicating existence of the target analyte. In one aspect, contacting the one or more surfaces or solutions with the system comprises spraying the system onto the one or more surfaces or adding the system to the solution. In another aspect, detecting the color change on the one or more surfaces or solutions comprises detecting a red-to-blue color change when the target analyte is indicated. In another aspect, detecting emergence of the color wavelength on the one or more surfaces or solutions comprises detecting emergence of a blue color wavelength in a range of 600-750 nm when the target analyte is indicated. In another aspect, detecting the color change on the one or more surfaces or solutions comprises detecting an absorbance at wavelengths of 600-750 nm and 520-540 nm and calculating a ratio of the absorbances (A₆₀₀₋₇₅₀/A₅₂₀₋₅₄₀) that is ≥0.2 when the target analyte is indicated. In another aspect, detecting the color change on the one or more surfaces or solutions comprises detecting an absorbance at wavelengths of 650 nm and 524 nm and calculating an absorbance ratio of (A₆₅₀/A₅₂₄) that is ≥0.2 when the target analyte is indicated. In another aspect, the ratio of the absorbances (A₆₀₀₋₇₅₀/A₅₂₀₋₅₄₀) is ≥0.8 when the target analyte is indicated. In another aspect, the method has a limit of detection for the target analyte of about 0.5 μg/mL to about 5 μg/mL. In another aspect, the method has a limit of detection for the target analyte of less than about 0.5 μg/mL. In another aspect, the method is performed in under 10 minutes.

Another embodiment described herein is a method for colorimetric detection of an opioid, the method comprising: contacting one or more surfaces or solutions with a system comprising: plasmonic gold nanoparticles (GNPs); an opioid-binding receptor conjugated or adsorbed to a surface of the GNPs; and a polymer-ligand conjugate comprising a polymer conjugated to an opioid ligand; and detecting emergence of a blue color wavelength in a range of 600-750 nm or detecting a red-to-blue color change on the one or more surfaces or solutions, indicating existence of the opioid. In one aspect, the opioid-binding receptor is a water-soluble mu-opioid receptor (wsMOR). In another aspect, the opioid is one or more of acetorphine; acetyl fentanyl; acetyldihydrocodeine; acetylmethadol; acetyl-α-methylfentanyl; acryl fentanyl; AH-7921; albuphine; allylprodine; alphacetylmethadol; benzethidine; benzylfentanyl; benzylmorphine; betacetylmethadol; betameprodine; betamethadol; beta-methyl fentanyl; betaprodine; buprenorphine; butorphanol; butyryl fentanyl; carfentanil; clonitazene; cocaine; codeine; codeine methylbromide; codeine-N-oxide; cyclopropyl fentanyl; cyprenorphine; desomorphine; dextromoramide; dextrorphan; diampromide; diethylthiambutene; difenoxin; dihydromorphine; dimenoxadol; dimepheptanol; dimethylthiambutene; dioxaphetyl butyrate; dipipanone; drotebanol; ethylmethylthiambutene; etonitazene; etorphine; etoxeridine; fentanyl carbamate; 2′-fluoro ortho-fluorofentanyl; 4-fluoroisobutyryl fentanyl; furanyl fentanyl; furethidine; heroin; hydrocodone; hydromorphinol; hydromorphone; β-hydroxy-3-methylfentanyl; β-hydroxyfentanyl; hydroxypethidine; β-hydroxythiofentanyl; ketobemidone; levomoramide; levophenacylmorphan; levorphanol; meperidine; methadone; methoxyacetyl fentanyl; methyldesorphine; methyldihydromorphine; α-meprodine; α-methadol; α-methylfentanyl; α-methylthiofentanyl; 1-methyl-4-phenyl-4-propionoxypiperidine; 3-methylfentanyl; 3-methylthiofentanyl; 4′-methyl acetyl fentanyl; morpheridine; morphine; morphine methylbromide; morphine methylsulfonate; morphine-N-oxide; MT-45; myrophine; nicocodeine; nicomorphine; noracymethadol; norlevorphanol; normethadone; normorphine; norpipanone; ocfentanil; ortho-fluoroacryl fentanyl; ortho-fluorobutyryl fentanyl; ortho-fluorofentanyl; ortho-fluoroisobutyryl fentanyl; ortho-methyl acetylfentanyl; ortho-methyl methoxyacetyl; oxycodone; oxymorphone; para-fluorobutyryl fentanyl; para-fluorofentanyl; para-methylfentanyl (4-methylfentanyl); pentazocine; phenadoxone; phenampromide; phenomorphan; phenoperidine; phenyl fentanyl (benzoyl fentanyl); β-phenyl fentanyl; 1-(-2-phenethyl)-4-phenyl-4-acetoxypiperidine (PEPAP); phoclodine; piritramide; proheptazine; properidine; propiram; propoxyphene; racemoramide; remifentanil; sufentanil; thebacon; tapentadol; thenylfentanyl; thiofentanyl; thiofuranyl fentanyl; tilidine; tramadol; trimeperidine; or U-47700. In another aspect, the polymer is polyethylene glycol (PEG) having a molecular weight >1000 Da and the opioid ligand is naloxone. In another aspect, the polymer is PEG having a molecular weight >1500 Da, >2000 Da, >2500 Da, >3000 Da, >3500 Da, >4000 Da, >4500 Da, or >5000 Da. In another aspect, the polymer is PEG having a molecular weight >3500 Da. In another aspect, the polymer is PEG having a molecular weight >5000 Da. In another aspect, the polymer is PEG having a molecular weight ranging from about 1000 Da to about 5000 Da. In another aspect, the polymer is PEG having a molecular weight ranging from about 1000 Da to about 3500 Da. In another aspect, the polymer is PEG having a molecular weight ranging from about 3500 Da to about 5000 Da. In another aspect, the polymer is PEG having a structure of:

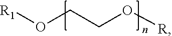

wherein R₁ is the ligand, R is H or C₁₋₄alkyl or hydroxyalkyl,

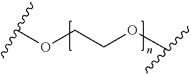

is the polyethylene glycol repeating unit (molecular weight of ˜44 g/mol), and n is 10 to 11000, including all integers and subranges within the specified range. In one aspect, n is 10-12000 (e.g., PEG ˜440-53000 Da), 10-100, 10-500, 10-1000, 10-5000, 10-10000, 100-1000, 100-1000, 100-10000, 500-1000, 500-10000, 1000-10000, 5000-10000, including all integers and subranges within the specified ranges. In another aspect, the polymer-ligand conjugate is:

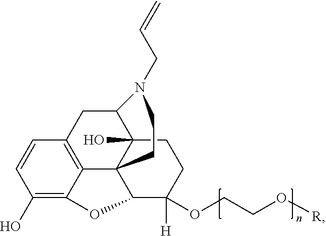

wherein R is H or C₁₋₄alkyl or hydroxyalkyl, and n is 10 to 11000. In another aspect, the system further comprises one or more salts selected from sodium chloride, potassium chloride, sodium phosphate, potassium phosphate, sodium bromide, sodium fluoride, or combinations thereof.

Another embodiment described herein is the use of a colorimetric detection system as a reagent, research tool, or forensics tool for colorimetric detection of a target analyte, the system comprising: plasmonic gold nanoparticles (GNPs); a binding receptor conjugated or adsorbed to a surface of the GNPs; a polymer-ligand conjugate; and one or more salts.

Another embodiment described herein is a kit for colorimetric detection of a target analyte, the kit comprising: a system comprising: plasmonic gold nanoparticles (GNPs); a binding receptor conjugated or adsorbed to a surface of the GNPs; a polymer-ligand conjugate; and one or more salts; a system applicator; optionally, a detection device; optionally, one or more articles of personal protective equipment; optionally, one or more buffers or receptacles; and optionally, one or more of packaging or instructions for use.

Another embodiment described herein is a system for colorimetric detection of a biotin-conjugated target analyte or free biotin, the system comprising: plasmonic gold nanoparticles (GNPs); streptavidin conjugated or adsorbed to a surface of the GNPs; a polymer-ligand conjugate comprising polyethylene glycol (PEG) conjugated to biotin; and about 0.1 M to about 0.3 M of one or more salts.

Another embodiment described herein is a method for colorimetric detection of a biotin-conjugated target analyte, the method comprising: contacting one or more surfaces or solutions with a system comprising: plasmonic gold nanoparticles (GNPs); strepavidin conjugated or adsorbed to a surface of the GNPs; and a polymer-biotin conjugate; and detecting a color change on the one or more surfaces or solutions, indicating existence of the biotin-conjugated target analyte.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described.

The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed.

Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A system for colorimetric detection of a target analyte, the system comprising:
   plasmonic gold nanoparticles (GNPs);
   a binding receptor conjugated or adsorbed to a surface of the GNPs; and
   a polymer-ligand conjugate.
Clause 2. The system of clause 1, wherein the binding receptor is an opioid-binding receptor.
Clause 3. The system of clause 1 or 2, wherein the opioid-binding receptor is a water-soluble mu-opioid receptor (wsMOR).
Clause 4. The system of any one of clauses 1-3, wherein the target analyte is selected from acetorphine; acetyl fentanyl; acetyldihydrocodeine; acetylmethadol; acetyl-α-methylfentanyl; acryl fentanyl; AH-7921; albuphine; allylprodine; alphacetylmethadol; benzethidine; benzylfentanyl; benzylmorphine; betacetylmethadol; betameprodine; betamethadol; beta-methyl fentanyl; betaprodine; buprenorphine; butorphanol; butyryl fentanyl; carfentanil; clonitazene; cocaine; codeine; codeine methylbromide; codeine-N-oxide; cyclopropyl fentanyl; cyprenorphine; desomorphine; dextromoramide; dextrorphan; diampromide; diethylthiambutene; difenoxin; dihydromorphine; dimenoxadol; dimepheptanol; dimethylthiambutene; dioxaphetyl butyrate; dipipanone; drotebanol; ethylmethylthiambutene; etonitazene; etorphine; etoxeridine; fentanyl carbamate; 2′-fluoro ortho-fluorofentanyl; 4-fluoroisobutyryl fentanyl; furanyl fentanyl; furethidine; heroin; hydrocodone; hydromorphinol; hydromorphone; β-hydroxy-3-methylfentanyl; β-hydroxyfentanyl; hydroxypethidine; β-hydroxythiofentanyl; ketobemidone; levomoramide; levophenacylmorphan; levorphanol; meperidine; methadone; methoxyacetyl fentanyl; methyldesorphine; methyldihydromorphine; α-meprodine; α-methadol; α-methylfentanyl; α-methylthiofentanyl; 1-methyl-4-phenyl-4-propionoxypiperidine; 3-methylfentanyl; 3-methylthiofentanyl; 4′-methyl acetyl fentanyl; morpheridine; morphine; morphine methylbromide; morphine methylsulfonate; morphine-N-oxide; MT-45; myrophine; nicocodeine; nicomorphine; noracymethadol; norlevorphanol; normethadone; normorphine; norpipanone; ocfentanil; ortho-fluoroacryl fentanyl; ortho-fluorobutyryl fentanyl; ortho-fluorofentanyl; ortho-fluoroisobutyryl fentanyl; ortho-methyl acetylfentanyl; ortho-methyl methoxyacetyl; oxycodone; oxymorphone; para-fluorobutyryl fentanyl; para-fluorofentanyl; para-methylfentanyl (4-methylfentanyl); pentazocine; phenadoxone; phenampromide; phenomorphan; phenoperidine; phenyl fentanyl (benzoyl fentanyl); β-phenyl fentanyl; 1-(-2-phenethyl)-4-phenyl-4-acetoxypiperidine (PEPAP); phoclodine; piritramide; proheptazine; properidine; propiram; propoxyphene; racemoramide; remifentanil; sufentanil; thebacon; tapentadol; thenylfentanyl; thiofentanyl; thiofuranyl fentanyl; tilidine; tramadol; trimeperidine; U-47700; or combinations thereof.
Clause 5. The system of any one of clauses 1-4, wherein the polymer is a polymer that stabilizes dispersion of the GNPs in a solution.
Clause 6. The system of any one of clauses 1-5, wherein the polymer-ligand conjugate comprises:

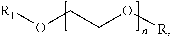

wherein R₁ is the ligand, R is H or C₁₋₄alkyl or hydroxyalkyl, and n is 10 to 11000.
Clause 7. The system of any one of clauses 1-6, wherein the ligand is selected from acetorphine; acetyl fentanyl; acetyldihydrocodeine; acetylmethadol; acetyl-α-methylfentanyl; acryl fentanyl; AH-7921; albuphine; allylprodine; alphacetylmethadol; benzethidine; benzylfentanyl; benzylmorphine; betacetylmethadol; betameprodine; betamethadol; beta-methyl fentanyl; betaprodine; buprenorphine; butorphanol; butyryl fentanyl; carfentanil; clonitazene; cocaine; codeine; codeine methylbromide; codeine-N-oxide; cyclopropyl fentanyl; cyprenorphine; desomorphine; dextromoramide; dextrorphan; diampromide; diethylthiambutene; difenoxin; dihydromorphine; dimenoxadol; dimepheptanol; dimethylthiambutene; dioxaphetyl butyrate; dipipanone; drotebanol; ethylmethylthiambutene; etonitazene; etorphine; etoxeridine; fentanyl carbamate; 2′-fluoro ortho-fluorofentanyl; 4-fluoroisobutyryl fentanyl; furanyl fentanyl; furethidine; heroin; hydrocodone; hydromorphinol; hydromorphone; β-hydroxy-3-methylfentanyl; β-hydroxyfentanyl; hydroxypethidine; β-hydroxythiofentanyl; ketobemidone; levomoramide; levophenacylmorphan; levorphanol; meperidine; methadone; methoxyacetyl fentanyl; methyldesorphine; methyldihydromorphine; α-meprodine; α-methadol; α-methylfentanyl; α-methylthiofentanyl; 1-methyl-4-phenyl-4-propionoxypiperidine; 3-methylfentanyl; 3-methylthiofentanyl; 4′-methyl acetyl fentanyl; morpheridine; morpheridine; morphine; morphine methylbromide; morphine methylsulfonate; morphine-N-oxide; MT-45; myrophine; nalmefene; naloxone; naltrexone; methylnaltrexone; nicocodeine; nicomorphine; noracymethadol; norlevorphanol; normethadone; normorphine; norpipanone; ocfentanil; ortho-fluoroacryl fentanyl; ortho-fluorobutyryl fentanyl; ortho-fluorofentanyl; ortho-fluoroisobutyryl fentanyl; ortho-methyl acetylfentanyl; ortho-methyl methoxyacetyl; oxycodone; oxymorphone; para-fluorobutyryl fentanyl; para-fluorofentanyl; para-methylfentanyl (4-methylfentanyl); pentazocine; phenadoxone; phenampromide; phenomorphan; phenoperidine; phenyl fentanyl (benzoyl fentanyl); β-phenyl fentanyl; 1-(-2-phenethyl)-4-phenyl-4-acetoxypiperidine (PEPAP); phoclodine; piritramide; proheptazine; properidine; propiram; propoxyphene; racemoramide; remifentanil; sufentanil; thebacon; tapentadol; thenylfentanyl; thiofentanyl; thiofuranyl fentanyl; tilidine; tramadol; trimeperidine; U-47700; or combinations thereof.
Clause 8. The system of any one of clauses 1-7, wherein the ligand is naloxone.
Clause 9. The system of any one of clauses 1-8, wherein the polymer-ligand conjugate comprises:

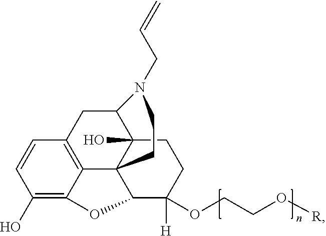

wherein R is H or C₁₋₄alkyl or hydroxyalkyl, and n is 10 to 11000.
Clause 10. The system of any one of clauses 1-9, further comprising one or more salts, buffers, organic additives, or a combination thereof.
Clause 11. The system of any one of clauses 1-10, wherein the one or more salts comprise sodium chloride, potassium chloride, sodium phosphate, potassium phosphate, sodium bromide, sodium fluoride, or combinations thereof.
Clause 12. The system of any one of clauses 1-11, wherein the one or more organic additives comprise one or more of benzyl mercaptan, urea, thiourea, cysteamine hydrochloride, tris(hydroxymethyl)-aminomethane (Tris), ethanol amine (EtA), glutathione (GSH), or poly(N-isopropylacrylamide) (PNIPAM).
Clause 13. The system of any one of clauses 1-12, wherein the one or more salts are present at a concentration of about 0.05 M to about 2.0 M.
Clause 14. The system of any one of clauses 1-13, wherein the one or more salts are present at a concentration of about 0.1 M to about 0.3 M.
Clause 15. The system of any one of clauses 1-14, wherein a ratio of GNPs:binding receptor:conjugate is about 1:5:30 to about 1:1000:4000.
Clause 16. The system of any one of clauses 1-15, further comprising one or more color masking agents, preservatives, surfactants, viscosifying agents, rheological modifying agents, thickening agents, anti-sagging agents, anti-freezing agents, solvents, or combinations thereof.
Clause 17. The system of any one of clauses 1-16, wherein the system is a solid or a liquid.
Clause 18. The system of any one of clauses 1-17, wherein the system is stable at room temperature for at least about 4 months as a solid.
Clause 19. A system for colorimetric detection of an opioid, the system comprising: plasmonic gold nanoparticles (GNPs);
   a water-soluble mu-opioid receptor (wsMOR) conjugated or adsorbed to a surface of the GNPs;
   a polymer-ligand conjugate comprising polyethylene glycol (PEG) conjugated to naloxone; and
   about 0.05 M to about 2.0 M of one or more salts.
Clause 20. A method for colorimetric detection of a target analyte, the method comprising:
   contacting one or more surfaces or solutions with a system comprising:
      plasmonic gold nanoparticles (GNPs);
      a binding receptor conjugated or adsorbed to a surface of the GNPs; and
      a polymer-ligand conjugate; and
   detecting emergence of a color wavelength or detecting a color change on the one or more surfaces or solutions, indicating existence of the target analyte.
Clause 21. The method of clause 20, wherein contacting the one or more surfaces or solutions with the system comprises spraying the system onto the one or more surfaces or adding the system to the solution.
Clause 22. The method of clause 20 or 21, wherein detecting the color change on the one or more surfaces or solutions comprises detecting a red-to-blue color change when the target analyte is indicated.
Clause 23. The method of any one of clauses 20-22, wherein detecting emergence of the color wavelength on the one or more surfaces or solutions comprises detecting emergence of a blue color wavelength in a range of 600-750 nm when the target analyte is indicated.
Clause 24. The method of any one of clauses 20-23, wherein detecting the color change on the one or more surfaces or solutions comprises detecting an absorbance at wavelengths of 600-750 nm and 520-540 nm and calculating a ratio of the absorbances (A₆₀₀₋₇₅₀/A₅₂₀₋₅₄₀) that is ≥0.2 when the target analyte is indicated.
Clause 25. The method of any one of clauses 20-24, wherein detecting the color change on the one or more surfaces or solutions comprises detecting an absorbance at wavelengths of 650 nm and 524 nm and calculating an absorbance ratio of (A₆₅₀/A₅₂₄) that is ≥0.2 when the target analyte is indicated.
Clause 26. The method of any one of clauses 20-25, wherein the ratio of the absorbances (A₆₀₀₋₇₅₀/A₅₂₀₋₅₄₀) is ≥0.8 when the target analyte is indicated.
Clause 27. The method of any one of clauses 20-26, wherein the method has a limit of detection for the target analyte of about 0.5 μg/mL to about 5 μg/mL.
Clause 28. The method of any one of clauses 20-27, wherein the method has a limit of detection for the target analyte of less than about 0.5 μg/mL.
Clause 29. The method of any one of clauses 20-28, wherein the method is performed in under 10 minutes.
Clause 30. A method for colorimetric detection of an opioid, the method comprising:
   contacting one or more surfaces or solutions with a system comprising:
      plasmonic gold nanoparticles (GNPs);
      an opioid-binding receptor conjugated or adsorbed to a surface of the GNPs; and
      a polymer-ligand conjugate comprising a polymer conjugated to an opioid ligand; and
   detecting emergence of a blue color wavelength in a range of 600-750 nm or detecting a red-to-blue color change on the one or more surfaces or solutions, indicating existence of the opioid.
Clause 31. The method of clause 30, wherein the opioid-binding receptor is a water-soluble mu-opioid receptor (wsMOR).
Clause 32. The method of clause 30 or 31, wherein the opioid is one or more of acetorphine; acetyl fentanyl; acetyldihydrocodeine; acetylmethadol; acetyl-α-methylfentanyl; acryl fentanyl; AH-7921; albuphine; allylprodine; alphacetylmethadol; benzethidine; benzylfentanyl; benzylmorphine; betacetylmethadol; betameprodine; betamethadol; beta-methyl fentanyl; betaprodine; buprenorphine; butorphanol; butyryl fentanyl; carfentanil; clonitazene; cocaine; codeine; codeine methylbromide; codeine-N-oxide; cyclopropyl fentanyl; cyprenorphine; desomorphine; dextromoramide; dextrorphan; diampromide; diethylthiambutene; difenoxin; dihydromorphine; dimenoxadol; dimepheptanol; dimethylthiambutene; dioxaphetyl butyrate; dipipanone; drotebanol; ethylmethylthiambutene; etonitazene; etorphine; etoxeridine; fentanyl carbamate; 2′-fluoro ortho-fluorofentanyl; 4-fluoroisobutyryl fentanyl; furanyl fentanyl; furethidine; heroin; hydrocodone; hydromorphinol; hydromorphone; β-hydroxy-3-methylfentanyl; β-hydroxyfentanyl; hydroxypethidine; β-hydroxythiofentanyl; ketobemidone; levomoramide; levophenacylmorphan; levorphanol; meperidine; methadone; methoxyacetyl fentanyl; methyldesorphine; methyldihydromorphine; α-meprodine; α-methadol; α-methylfentanyl; α-methylthiofentanyl; 1-methyl-4-phenyl-4-propionoxypiperidine; 3-methylfentanyl; 3-methylthiofentanyl; 4′-methyl acetyl fentanyl; morpheridine; morphine; morphine methylbromide; morphine methylsulfonate; morphine-N-oxide; MT-45; myrophine; nicocodeine; nicomorphine; noracymethadol; norlevorphanol; normethadone; normorphine; norpipanone; ocfentanil; ortho-fluoroacryl fentanyl; ortho-fluorobutyryl fentanyl; ortho-fluorofentanyl; ortho-fluoroisobutyryl fentanyl; ortho-methyl acetylfentanyl; ortho-methyl methoxyacetyl; oxycodone; oxymorphone; para-fluorobutyryl fentanyl; para-fluorofentanyl; para-methylfentanyl (4-methylfentanyl); pentazocine; phenadoxone; phenampromide; phenomorphan; phenoperidine; phenyl fentanyl (benzoyl fentanyl); β-phenyl fentanyl; 1-(-2-phenethyl)-4-phenyl-4-acetoxypiperidine (PEPAP); phoclodine; piritramide; proheptazine; properidine; propiram; propoxyphene; racemoramide; remifentanil; sufentanil; thebacon; tapentadol; thenylfentanyl; thiofentanyl; thiofuranyl fentanyl; tilidine; tramadol; trimeperidine; or U-47700.
Clause 33. The method of any one of clauses 30-32, wherein the polymer is polyethylene glycol (PEG) having a molecular weight >1000 Da and the opioid ligand is naloxone.
Clause 34. The method of any one of clauses 30-33, wherein the polymer-ligand conjugate is:

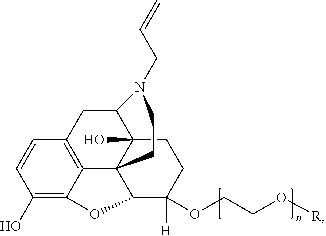

wherein R is H or C₁₋₄alkyl or hydroxyalkyl, and n is 10 to 11000.
Clause 35. The method of any one of clauses 30-34, wherein the system further comprises one or more salts selected from sodium chloride, potassium chloride, sodium phosphate, potassium phosphate, sodium bromide, sodium fluoride, or combinations thereof.
Clause 36. Use of a colorimetric detection system as a reagent, research tool, or forensics tool for colorimetric detection of a target analyte, the system comprising:
   plasmonic gold nanoparticles (GNPs);
   a binding receptor conjugated or adsorbed to a surface of the GNPs;
   a polymer-ligand conjugate; and
   one or more salts.
Clause 37. A kit for colorimetric detection of a target analyte, the kit comprising:
   a system comprising:
      plasmonic gold nanoparticles (GNPs);
      a binding receptor conjugated or adsorbed to a surface of the GNPs;
      a polymer-ligand conjugate; and
      one or more salts;
   a system applicator;
   optionally, a detection device;
   optionally, one or more articles of personal protective equipment;
   optionally, one or more buffers or receptacles; and
   optionally, one or more of packaging or instructions for use.

### EXAMPLES

### Example 1

### Synthesis of Gold Nanoparticles (GNPs)

GNPs were prepared by the classical Turkevich method in which chloroauric acid (HAuCl₄·3H₂O) reacts with trisodium citrate (Na₃C₆H₅O₇). See Dong, et al., Kona 37: 224-232 (2020), which is incorporated by reference herein for such teachings. The 25 nm GNPs were prepared with the molar ratio of HAuCl₄·3 H₂O:NaCl of 1:2.8. In a typical reaction, 83.4 mL 1.2 mM chloroauric acid (99.9%, Alfa Aesar) was boiled in a flask, and 7.78 mL 36 mM trisodium citrate (99.9%, Sigma-Aldrich) was added to the flask. The reaction was kept boiling for 30 minutes and then cooled down. After the synthesis reaction, the 25 nm GNPs were further purified and concentrated by centrifugation process (2000×g, 20 min). The concentrated GNPs will be diluted by using 1.8 mM K₂CO₃ buffer solution to the desired concentration for subsequent preparation. The plasmonic peak is centered at 524 nm. The OD value of the GNPs samples was determined by the absorption intensity of the plasmonic peak wavelength. The hydrodynamic particle size and Zeta potential was measured by Nano-ZS Zetasizer, Malvern. The hydrodynamic particle size is ˜33 nm, which is larger than the real size of 25 nm, which was confirmed by the TEM analysis conducted previously. The Zeta potential of the GNP suspension was −36 mV (in the range of −45 to −30 mV). The molar concentration of the GNP suspension is estimated around 5.4×10⁻¹⁰ M at OD of 1.0.

### Example 2

Expression and Purification of wsMOR

The water-soluble mu-opioid receptor (wsMOR) is an engineered variant of the transmembrane domain of the human mu-opioid receptor described in U.S. Pat. No. 9,670,264, which is incorporated by reference herein for such teachings. The polypeptide has a sequence as shown below and a molecular weight of 36 kDa.

Water-soluble mu-opioid receptor (wsMOR)
(SEQ ID NO: 1)
SMITAIKIHEEYKKVCEEGKKGNKLVMEVIVRYTKMKTATNIYIFNLAK
ADALAESTLPFQSVNKLMGTWPFGTILCKKVISIDYYNMFTSIFTLCTM
SVDRYIAVCHPVKALDERTPRNAKEENEKNWKLSSEIGKPVEKKATTKY
RQGSIDCTLTFSHPTWYWEDKLKDEVFKKAFEEPVKKIKECYGLMILRL
KSVRMLSGSKEKDRNLRRITRMVLVVVEVFIKCWTEIHKYVKEGKLVTI
PETTFQTVSWHECIAKGYKNSCENPKLYEELDENFKRCFREFC

The water-soluble MOR (wsMOR) was over-expressed in E. coli BL21(DE3) cells (EMD/Novagen) as described by Perez-Aguilar et al., PLOS ONE 8(6), e66009 (2013), which is incorporated by reference herein for such teachings. The sequences of the synthetic cDNA encodings of the water-soluble MOR were subcloned between the Nde I and Xho I restriction sites of the expression plasmid pET-28b(+) (EMD/Novagen). This cloning strategy results in placement of a His-tag at the amino terminus of the protein. E. coli BL21(DE3) cells were used for expression and the product was purified using nickel affinity chromatography.

### Example 3

### Synthesis of Naloxol-PEG, 5K

The synthesis of naloxone conjugated to low molecular weight polyethylene glycol HO—(CH₂CH₂O)ₙ—CH₃ moieties (<500 Da; PEG repeats of n=5-9) for use as therapeutics is described in U.S. Pat. Nos. 7,786,133; 8,034,825; 8,067,431; and 11,129,794, each of which are incorporated by reference herein for such teachings. Modifications of these synthesis methods were utilized for the preparation of higher molecular weight Naloxone-PEG conjugates.

Naloxol-PEG(5K) was prepared by the conjugation of naloxone with mPEG5K-Tosylate (TsO(CH₂CH₂O)ₙCH₃). The synthesis route is shown in the scheme below.

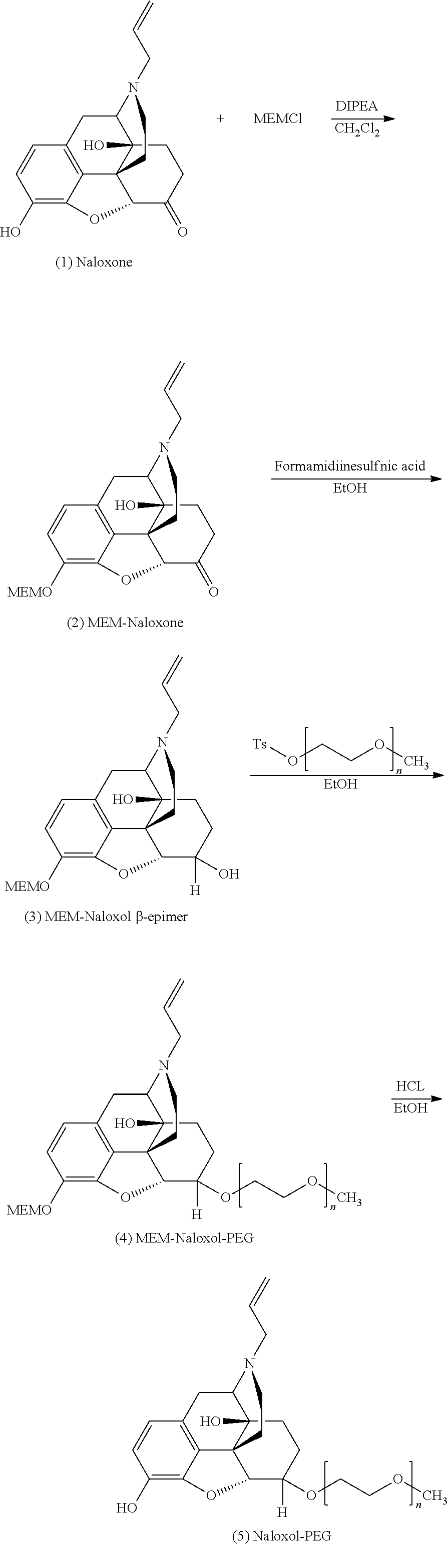

The stepwise synthetic method is described as follows.

### Synthesis of (4aS,7aR,12bS)-3-allyl-4a-hydroxy-9-((2-methoxyethoxy)methoxy)-2,3,4,4a,5,6-hexahydro-1H-4, 12-methanobenzofuro[3,2-e]isoquinolin-7(7aH)-one (2, MEM-Naloxone)

Naloxone hydrochloride 1 (1.83 g, 4.73 mmol) was added into a flask and dissolved in 30 mL of dichloromethane. Diisopropylethylamine (DIPEA, 3.05 g, 23.65 mmol) was added to the solution. The mixture was stirred for 15 mins before MEMCI (2.53 g, 20.34 mmol) was added dropwise to the solution. Then reaction was allowed to progress at room temperature for 30 h. Water was added three times (30 mL×3) for extraction. The organic phase was washed with saturated sodium chloride solution before it was dried over sodium sulfate. Concentration and purification by column chromatography offered product 2 (1.34 g, 68%).

### Synthesis of (4aS,7aR,12bS)-3-allyl-9-((2-methoxyethoxy)methoxy)-1,2,3,4,5,6,7,7a-octahydro-4aH-4, 12-methanobenzofuro[3,2-e]isoquinoline-4a,7-diol (3, MEM-Naloxol)

MEM-Naloxone (1.03 g, 2.5 mmol), aminoiminomethane sulfinic acid (1.11 g, 10.2 mmol), sodium hydroxide solution (0.6 M, 21 mL) were added into ethanol (21 mL). The reaction mixture was heated to 80° C. for stirring 2 h, and the mixture was cooled down to room temperature. Then 10% sodium chloride aqueous solution was added into above mixture, and the mixture was extracted with CH₂Cl₂ (50 mL×3), combined organic layer was washed with brine, dried over MgSO₄, concentrated. The residue was purified by silica gel column chromatography to afford product 3 (0.68 g, 66%).

### Synthesis of MEM-Naloxol-PEG (4)

Sodium hydride (60%, 8.0 mg, 0.2 mmol) was added into MEM-Naloxone (10 mg, 0.024 mmol) solution of DMF (1 mL). After 15 min., mPEG-Tosylate, MM 5K (0.02 mmol) solution in DMF (2 mL) was added. The reaction mixture was allowed to react at 30° C. overnight. Then the reaction mixture was cooled to RT, cold ether was poured into above solution, and the resulting precipitate was separated by centrifugal filtration. The crude product was dialyzed and lyophilized to yield a white solid product 4.

### Synthesis of Naloxol-PEG, 5K (5)

Compound 4 (30 mg) was dissolved in 2 mL of 0.5 M HCl-methanol solution. The mixture was stirred at 60° C. After the reaction was complete as monitored by HPLC, stirring was stopped. The mixture was concentrated, and the residue was dialyzed and lyophilized to obtain white solid product 5. The identity and purity (>90%) of the final products were confirmed by NMR. ¹H NMR (600 MHZ, D₂O) δ 6.75 (d, J=8.0 Hz, 1H), 6.56 (d, J=8.1 Hz, 1H), 5.32 (m, 2H), 5.07 (m, 1H), 4.54 (d, J=6.6 Hz, 1H), 4.19 (s, 1H), 3.62 (m, 460H), 3.38 (s, 3H), 3.33-3.02 (m, 2H), 2.38-2.22 (m, 2H), 2.06-1.87 (m, 3H), 1.78-1.63 (m, 2H), 1.52-1.27 (m, 4H).

### Example 4

### Synthesis of Naloxol-PEG, 10K

Sodium hydride (60%, 8.0 mg, 0.2 mmol) was added into MEM-Naloxol (10 mg, 0.024 mmol) solution of DMF (1 mL). After 15 min., mPEG-Tosylate, MW 10K (0.02 mmol) solution in DMF (2 mL) was added. The reaction mixture was allowed to react at 30° C. overnight. Then the reaction mixture was cooled to RT, cold ether was added, and thed resulting precipitate was separated by centrifugal filtration. The crude product was dialyzed and lyophilized to obtain a white solid product MEM-Naloxol-PEG(10K). 60 mg MEM-Naloxol-PEG(10K) was dissolved in dissolved in 2 mL of 0.5 M HCl methanol solution. The mixture was stirred at 60° C. After the reaction was complete as monitored by HPLC, stirring was stopped. The mixture was concentrated, and the residue was dialyzed and lyophilized to obtain a white solid product Naloxol-PEG, 10K. The identity and purity of the final products were confirmed by NMR. ¹H NMR (600 MHZ, D20) δ 6.73 (d, J=8.0 Hz, 1H), 6.57 (d, J=8.1 Hz, 1H), 5.32 (m, 2H), 5.09 (m, 1H), 4.51 (d, J=6.6 Hz, 1H), 4.13 (s, 1H), 3.65 (m, 950H), 3.34 (s, 3H), 3.30-3.04 (m, 2H), 2.33-2.26 (m, 2H), 2.08-1.87 (m, 3H), 1.78-1.62 (m, 2H), 1.54-1.23 (m, 4H).

### Example 5

### Synthesis of Naloxol-PEG, 20K

Sodium hydride (60%, 8.0 mg, 0.2 mmol) was added into MEM-Naloxol (10 mg, 0.024 mmol) solution of DMF (1 mL). After 15 min., mPEG-Tosylate, MW 20K (0.02 mmol) solution in DMF (2 mL) was added. The reaction mixture was allowed to react at 30° C. overnight. Then the reaction mixture was cooled to RT, cold ether was added, and thed resulting precipitate was separated by centrifugal filtration. The crude product was dialyzed and lyophilized to obtain a white solid product MEM-Naloxol-PEG(20K). 120 mg MEM-Naloxol-PEG(20K) was dissolved in dissolved in 2 mL of 0.5 M HCl methanol solution. The mixture was stirred at 60° C. After complete reaction monitored by HPLC, stirring was stopped. The mixture was concentrated, and the residue was dialyzed and lyophilized to obtain a white solid product 5. The identity and purity of the final products were confirmed by NMR. ¹H NMR (600 MHZ, D₂O) δ 6.74 (d, J=8.0 Hz, 1H), 6.58 (d, J=8.1 Hz, 1H), 5.34 (m, 2H), 5.08 (m, 1H), 4.53 (d, J=6.6 Hz, 1H), 4.17 (s, 1H), 3.64 (m, 1950H), 3.38 (s, 3H), 3.30-3.00 (m, 2H), 2.38-2.26 (m, 2H), 2.04-1.88 (m, 3H), 1.78-1.61 (m, 2H), 1.55-1.27 (m, 4H).

### Example 6

### Preparation of Opioid Detection Solution—[GNP:MOR:Naloxol-PEG 10K=1:20:400]

The preparation procedure of the opioid detection formulation [GNP/MOR/Naloxol-PEG] includes three steps: in the first step, the MOR receptor solution is reacted with the Naloxol-PEG to form uniform complex compound solution. The second step, the complex compound solution reacts with GNP suspension. The receptors (ws-MOR) anchored on the GNP surface forming a well aligned nanostructure in which the Naloxol-PEGs are outer layer with their PEG tails toward outside. In the third step, NaCl is added to the mixture. Below is the detailed process to prepare a detection solution with formulation of [GNP/MOR/Naloxol-PEG].

MOR (MW: 36 KDa) solution with a concentration of 2.78 UM was prepared by dissolving MOR dry powder to 1.8 mM K₂CO₃ buffer. Naloxol-PEG 10K solution with a concentration of 19.2 μM was prepared by dissolving the Naloxol-PEG 10K dry powder to 1.8 mM K₂CO₃ buffer. The concentrated GNP suspension was adjusted to a concentration of 5 OD by using 1.8 mM K₂CO₃ buffer. In the first step, the 2.78 μM MOR solution was mixed with the 19.2 μM Naloxol-PEG10K solution in a 2 mL tube and vortexed for 2 min. The MOR/Naloxol-PEG complex compound was formed in the mixture. In the second step, 1 mL of 25 nm GNP solution with concentration of 2.7 nM (5 OD) was added to the MOR/Naloxol-PEG complex compound, vortexed for 2 min, and left to react for 4 min. In the third step. 186 μL NaCl solution was added to the mixture, and vortexed for 2 min. This process generated an opioid detection solution of [GNP/MOR/Naloxol-PEG10K] with molar ratio of [GNP:MOR:Naloxol-PEG 10K=1:20:400] in 0.30 M NaCl solution, which was used for opioid detection.

### Example 7

### Preparation of Opioid Detection Solution—[GNP:MOR:Naloxol-PEG 5K=1:20:1100]

In the first step, 53.9 μL of 1.0 μM MOR solution was mixed with 297 μL of 10 UM Naloxol-PEG10K solution in a 15 mL testing tube and vortexed for 2 min. The MOR/Naloxol-PEG complex compound formed in the mixture. In the second step, 5 mL of 25 nm GNP solution with a concentration of 0.54 nM (1 OD) was added to the MOR/Naloxol-PEG complex compound, vortexed for 2 min, and left to react for 4 min. In the third step. 382 μL of 3 M NaCl solution was added to the mixture, and vortex for 2 min. This process generated an opioid detection solution of [GNP/MOR/Naloxol-PEG 5K] with molar ratio of [GNP:MOR:Naloxol-PEG 5K=1:20:1100] in 0.20 M NaCl solution, which was used for opioid detection.

### Example 8

### Detection of Fentanyl and its Derivatives

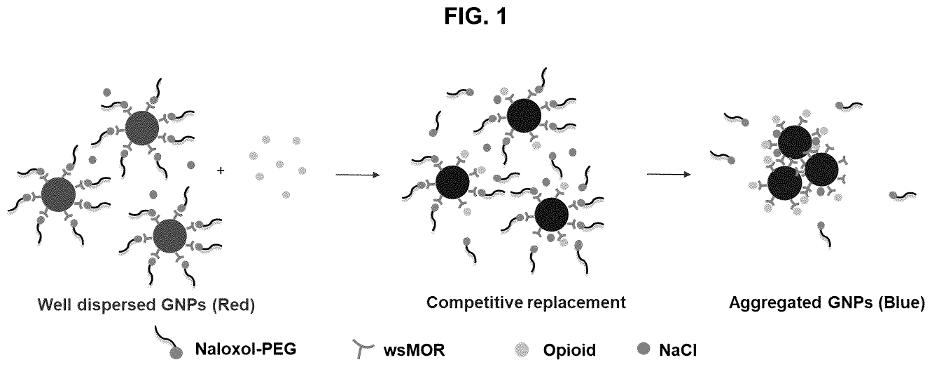

The detection method described herein provides a simple and highly selective colorimetric method for the detection of opioid compounds, especially for the potent synthetic opioids including fentanyl and its derivatives. FIG. 1 shows the detection mechanism. The solution-based formulation is comprised of gold nanoparticles, water soluble mu-opioid receptors (wsMOR; SEQ ID NO: 1), the PEGylated Naloxone (Naloxone-PEG) and NaCl. The bioengineered, thermally stable wsMOR is able to bind and recognize a broad-spectrum class of opioids with high binding affinity (KD) at subnanomolar to nanomolar. Its binding affinity (K_{d}) to Naloxone (a synthetic opioid receptor antagonist) and fentanyl is 1.76 nM and 0.6 nM, respectively. As a result, wsMOR is able to form complex with Naloxone-PEG via the binding between wsMOR and Naloxone. The GNP surface is protected by wsMOR/Naloxone-PEG complexes in the system, where the N-PEG works as a spacer to maintain proper distance between GNPs, avoiding aggregation in the system. At desired molar ratio of each component in the system, as well as the proper length of the PEG spacer, the GNPs could be well dispersed and display stable wine-red color. In the presence of opioid compounds such as fentanyl and its derivatives, the Naloxone-PEG spacers are competitively replaced by the opioids, the well dispersed GNPs aggregate under the attack of the NaCl and result in the red to blue color change due to the plasmonic coupling effect.

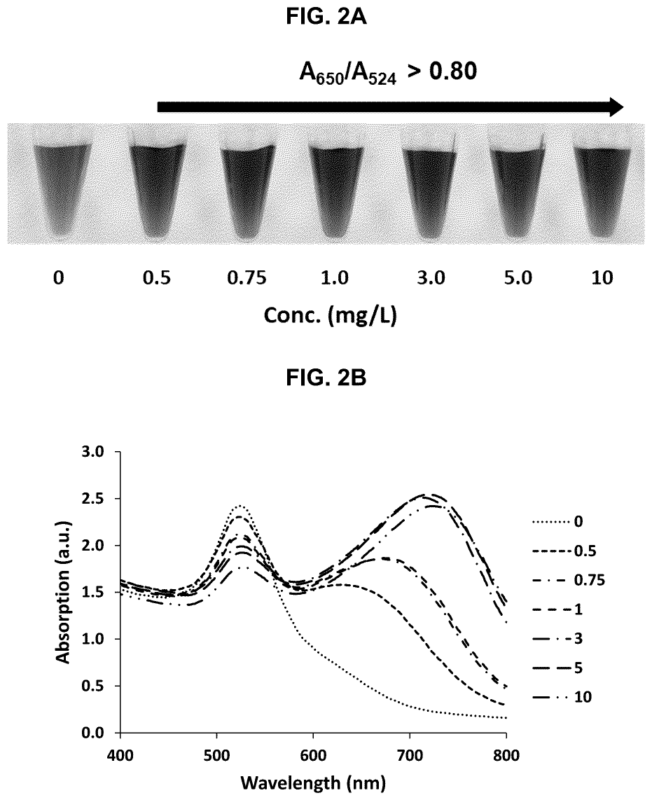

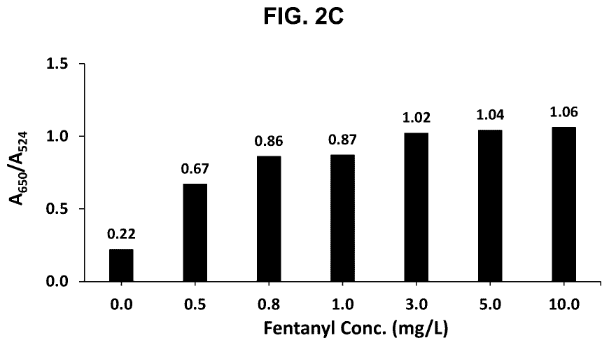

FIG. 2A-B show the detection of fentanyl in solution with the optimized detection solution. In FIG. 2A, the blank detection solution (0 mg/L fentanyl) shows the wine-red color, which is related to the quadrupole plasmon excitation at 524 nm in the curve of blank detection solution in FIG. 2B. When fentanyl analyte was added to the detection solution, the color turns to purple and blue gradually with increasing analyte concentrations. In the spectra shown in FIG. 2B, the plasmonic peak of the detection solution at 524 nm drops and the second dipole plasmonic peak rises in the range of 650 nm to 750 nm, associated with the blue color change. This is resulted from the aggregation of the AuNPs. FIG. 2C shows the absorbance ratio of 650 nm and 524 nm (A₆₅₀/A₅₂₄), which is an established visual red to blue color change indicator. The limit of detection (LOD) of this formulation, as defined as the concentration that reaches A₆₅₀/A₅₂₄=0.8 in 10 minutes, is 0.67 mg/L (Table 1).

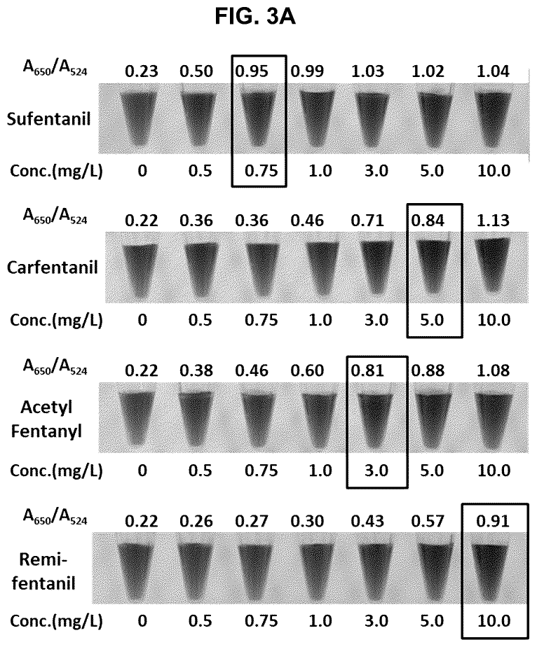

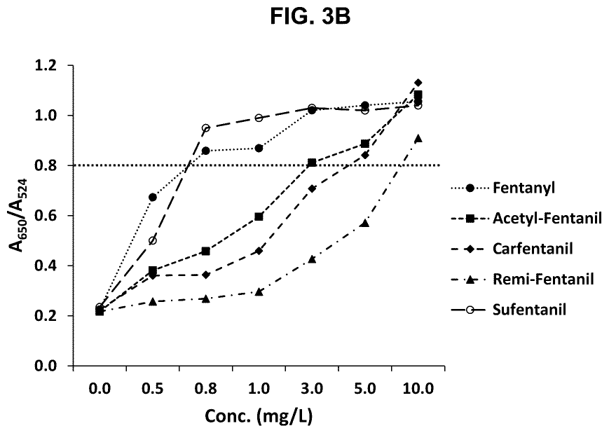

The detection system also showed the capability to detect various fentanyl derivatives including sufentanil, carfentanil, remifentanil, and acetylfentanyl. The photos in FIG. 3A recorded the color of the solutions at 10 mins after addition the analytes into detection solution. FIG. 3B shows the A₆₅₀/A₅₂₄ ratio as a function of the analyte concentration and compares the ratio of fentanyl derivatives with fentanyl (from FIG. 2A). The red dash line marks the A₆₅₀/A₅₂₄ ratio of 0.80. The LOD of the analytes which were determined from the intersection of the A₆₅₀/A₅₂₄ ratio curve are listed in Table 1.

TABLE 1
Limit of Detection of Fentanyl and its Derivatives

| Opioids | LOD (mg/L) |
|---|---|
| Fentanyl | 0.67 |
| Sufentanil | 0.67 |
| Carfentanil | 4.37 |
| Acetylfentanyl | 2.90 |
| Remifentanil | ≤4.20* |

*Corrected LOD given that the purity of the analyzed remifentanil is ≤50%.

### Example 9

### Detection of Fentanyl Under Harsh Environments

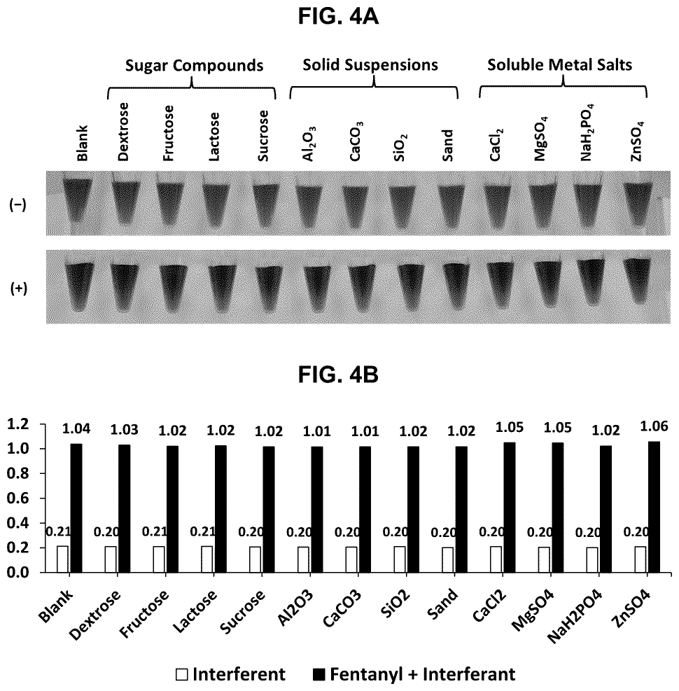

The detection system showed excellent tolerance to a broad species of potential interferents, including organic compounds (sugars including dextrose, fructose, lactose, and sucrose), solid suspensions (Al₂O₃, CaCO₃, SiO₂ and sand fine particles) and inorganic salts (CaCl₂), MgSO₄, NaH₂PO₄, and ZnSO₄). As the photographs in FIG. 4A show, the detection solution remained red in the presence of 500 mg/L of single interferent compound and turned into purple/blue when 10 mg/L of fentanyl was added together. The absorbance ratio A₆₅₀/A₅₂₄ of each sample is presented below their photos (FIG. 4B).

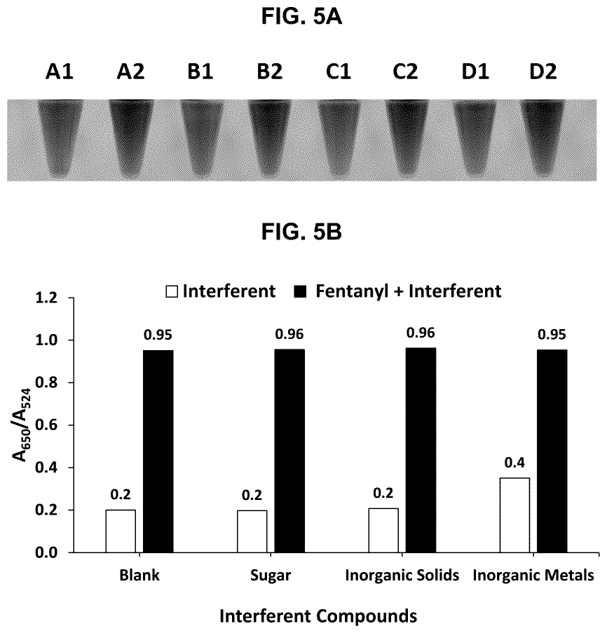

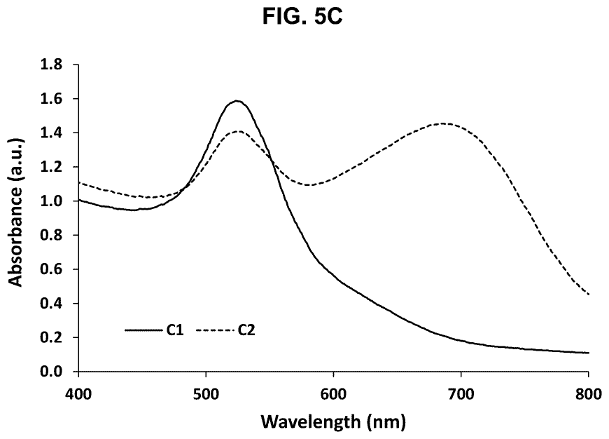

The tolerance of the detection solution was tested with interference compounds. As shown in FIG. 5A, no color change was observed in the presence of a mixture of four sugars (dextrose, fructose, lactose, and sucrose), four inorganic solid particles (Al₂O₃, CaCO₃, SiO₂, and sand fine particles) or four inorganic metal salts (CaCl₂), MgSO₄, NaH₂PO₄, and ZnSO₄) at the concentration of 2 g/L (500 mg/L of each). While in the presence of 10 mg/L fentanyl, an obvious color change was observed whether there were interferent compounds in the solution or not, which demonstrated excellent selectivity of the opioid detection formulation to opioid compounds. The result was also confirmed by the A₆₅₀/A₅₂₄ ratios (FIG. 5B). In such high interferent concentration, the detection solution with sugar mixture (B1) and the solid inorganic particle mixture (C1) present very close A₆₅₀/A₅₂₄ ratio value to that of the blank detection solution (A1), indicating that these interferent mixtures did not affect the fentanyl detection. Although the detection solution inorganic metal salts mixture has higher A₆₅₀/A₅₂₄ ratio (0.35), naked-eye visible color change in the presence of fentanyl can still be well observed. The spectra of the mixtures of inorganic solid suspension interferents is shown in FIG. 5C.

### Example 10

### Detection of Biotin

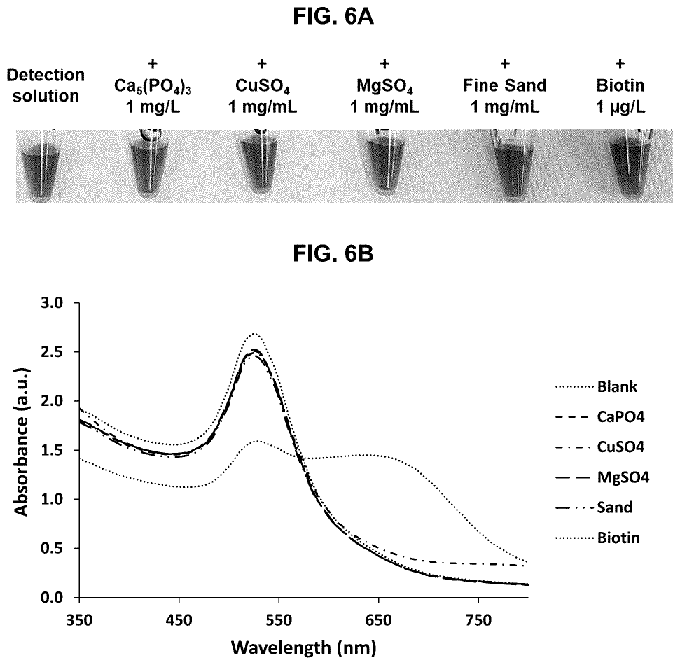

The interaction between the biotin and Streptavidin (SAV) is one of the strongest non-covalent binding interactions; the binding affinity is about 10⁻¹⁴ M, which is even higher than many antibodies binding to antigens. To demonstrate the feasibility and adaptability of the detection platform for the detection of any ligand based on the competitive replacement of the PEGylated ligands from its corresponding receptor, a detection system was developed that consists of the GNPs, streptavidin, PEGylated biotin (biotin-PEG) and NaCl for the detection of free biotin in solution. As FIG. 6A shows, the detection solution specifically responds to the biotin target with naked eye visible red to blue color changes at the biotin concentration 10 mg/L.

No responses to the interferent compounds including fine sand suspensions, CuSO₄, MgSO₄, and Ca₃(PO₄)₅ (1000 mg/L) were observed. See FIG. 6A-6B.

1. A system for colorimetric detection of a target analyte, the system comprising:
plasmonic gold nanoparticles (GNPs);
a binding receptor conjugated or adsorbed to a surface of the GNPs; and
a polymer-ligand conjugate, wherein the polymer is a polymer that stabilizes dispersion of the GNPs in a solution.

2. The system of claim 1, wherein the binding receptor is an opioid-binding receptor.

3. The system of claim 2, wherein the opioid-binding receptor is a water-soluble mu-opioid receptor (wsMOR).

4. The system of claim 1, wherein the target analyte is selected from acetorphine; acetyl fentanyl; acetyldihydrocodeine; acetylmethadol; acetyl-α-methylfentanyl; acryl fentanyl; AH-7921; albuphine; allylprodine; alphacetylmethadol; benzethidine; benzylfentanyl; benzylmorphine; betacetylmethadol; betameprodine; betamethadol; beta-methyl fentanyl; betaprodine; buprenorphine; butorphanol; butyryl fentanyl; carfentanil; clonitazene; cocaine; codeine; codeine methylbromide; codeine-N-oxide; cyclopropyl fentanyl; cyprenorphine; desomorphine; dextromoramide; dextrorphan; diampromide; diethylthiambutene; difenoxin; dihydromorphine; dimenoxadol; dimepheptanol; dimethylthiambutene; dioxaphetyl butyrate; dipipanone; drotebanol; ethylmethylthiambutene; etonitazene; etorphine; etoxeridine; fentanyl carbamate; 2′-fluoro ortho-fluorofentanyl; 4-fluoroisobutyryl fentanyl; furanyl fentanyl; furethidine; heroin; hydrocodone; hydromorphinol; hydromorphone; β-hydroxy-3-methylfentanyl; β-hydroxyfentanyl; hydroxypethidine; β-hydroxythiofentanyl; ketobemidone; levomoramide; levophenacylmorphan; levorphanol; meperidine; methadone; methoxyacetyl fentanyl; methyldesorphine; methyldihydromorphine; α-meprodine; α-methadol; α-methylfentanyl; α-methylthiofentanyl; 1-methyl-4-phenyl-4-propionoxypiperidine; 3-methylfentanyl; 3-methylthiofentanyl; 4′-methyl acetyl fentanyl; morpheridine; morphine; morphine methylbromide; morphine methylsulfonate; morphine-N-oxide; MT-45; myrophine; nicocodeine; nicomorphine; noracymethadol; norlevorphanol; normethadone; normorphine; norpipanone; ocfentanil; ortho-fluoroacryl fentanyl; ortho-fluorobutyryl fentanyl; ortho-fluorofentanyl; ortho-fluoroisobutyryl fentanyl; ortho-methyl acetylfentanyl; ortho-methyl methoxyacetyl; oxycodone; oxymorphone; para-fluorobutyryl fentanyl; para-fluorofentanyl; para-methylfentanyl (4-methylfentanyl); pentazocine; phenadoxone; phenampromide; phenomorphan; phenoperidine; phenyl fentanyl (benzoyl fentanyl); β-phenyl fentanyl; 1-(-2-phenethyl)-4-phenyl-4-acetoxypiperidine (PEPAP); phoclodine; piritramide; proheptazine; properidine; propiram; propoxyphene; racemoramide; remifentanil; sufentanil; thebacon; tapentadol; thenylfentanyl; thiofentanyl; thiofuranyl fentanyl; tilidine; tramadol; trimeperidine; U-47700; or combinations thereof.

5. (canceled)

6. The system of claim 1, wherein the polymer-ligand conjugate comprises: 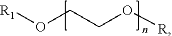
wherein R₁ is the ligand, R is H or C₁₋₄alkyl or hydroxyalkyl, and n is 10 to 11000.

7. The system of claim 1, wherein the ligand is selected from acetorphine; acetyl fentanyl; acetyldihydrocodeine; acetylmethadol; acetyl-α-methylfentanyl; acryl fentanyl; AH-7921; albuphine; allylprodine; alphacetylmethadol; benzethidine; benzylfentanyl; benzylmorphine; betacetylmethadol; betameprodine; betamethadol; beta-methyl fentanyl; betaprodine; buprenorphine; butorphanol; butyryl fentanyl; carfentanil; clonitazene; cocaine; codeine; codeine methylbromide; codeine-N-oxide; cyclopropyl fentanyl; cyprenorphine; desomorphine; dextromoramide; dextrorphan; diampromide; diethylthiambutene; difenoxin; dihydromorphine; dimenoxadol; dimepheptanol; dimethylthiambutene; dioxaphetyl butyrate; dipipanone; drotebanol; ethylmethylthiambutene; etonitazene; etorphine; etoxeridine; fentanyl carbamate; 2′-fluoro ortho-fluorofentanyl; 4-fluoroisobutyryl fentanyl; furanyl fentanyl; furethidine; heroin; hydrocodone; hydromorphinol; hydromorphone; β-hydroxy-3-methylfentanyl; β-hydroxyfentanyl; hydroxypethidine; β-hydroxythiofentanyl; ketobemidone; levomoramide; levophenacylmorphan; levorphanol; meperidine; methadone; methoxyacetyl fentanyl; methyldesorphine; methyldihydromorphine; α-meprodine; α-methadol; α-methylfentanyl; α-methylthiofentanyl; 1-methyl-4-phenyl-4-propionoxypiperidine; 3-methylfentanyl; 3-methylthiofentanyl; 4′-methyl acetyl fentanyl; morpheridine; morpheridine; morphine; morphine methylbromide; morphine methylsulfonate; morphine-N-oxide; MT-45; myrophine; nalmefene; naloxol; naloxone; naltrexone; methylnaltrexone; nicocodeine; nicomorphine; noracymethadol; norlevorphanol; normethadone; normorphine; norpipanone; ocfentanil; ortho-fluoroacryl fentanyl; ortho-fluorobutyryl fentanyl; ortho-fluorofentanyl; ortho-fluoroisobutyryl fentanyl; ortho-methyl acetylfentanyl; ortho-methyl methoxyacetyl; oxycodone; oxymorphone; para-fluorobutyryl fentanyl; para-fluorofentanyl; para-methylfentanyl (4-methylfentanyl); pentazocine; phenadoxone; phenampromide; phenomorphan; phenoperidine; phenyl fentanyl (benzoyl fentanyl); β-phenyl fentanyl; 1-(-2-phenethyl)-4-phenyl-4-acetoxypiperidine (PEPAP); phoclodine; piritramide; proheptazine; properidine; propiram; propoxyphene; racemoramide; remifentanil; sufentanil; thebacon; tapentadol; thenylfentanyl; thiofentanyl; thiofuranyl fentanyl; tilidine; tramadol; trimeperidine; U-47700; or combinations thereof.

8. The system of claim 7, wherein the ligand is naloxone.

9. The system of claim 1, wherein the polymer-ligand conjugate comprises: 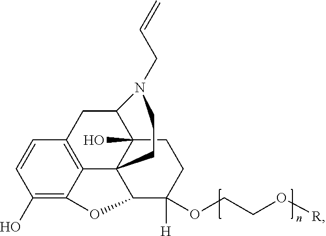
wherein R is H or C₁₋₄alkyl or hydroxyalkyl, and n is 10 to 11000.

10. The system of claim 1, further comprising one or more salts, buffers, organic additives, or a combination thereof.

11. The system of claim 10, wherein the one or more salts comprise sodium chloride, potassium chloride, sodium phosphate, potassium phosphate, sodium bromide, sodium fluoride, or combinations thereof.

12. The system of claim 10, wherein the one or more organic additives comprise one or more of benzyl mercaptan, urea, thiourea, cysteamine hydrochloride, tris(hydroxymethyl)-aminomethane (Tris), ethanol amine (EtA), glutathione (GSH), or poly(N-isopropylacrylamide) (PNIPAM).

13. The system of claim 10, wherein the one or more salts are present at a concentration of about 0.05 M to about 2.0 M.

14. The system of claim 13, wherein the one or more salts are present at a concentration of about 0.1 M to about 0.3 M.

15. The system of claim 1, wherein a ratio of GNPs:binding receptor:conjugate is about 1:5:30 to about 1:1000:4000.

16. The system of claim 1, further comprising one or more color masking agents, preservatives, surfactants, viscosifying agents, rheological modifying agents, thickening agents, anti-sagging agents, anti-freezing agents, solvents, or combinations thereof.

17. The system of claim 1, wherein the system is a solid or a liquid.

18. The system of claim 17, wherein the system is stable at room temperature for at least about 4 months as a solid.

19. (canceled)

20. A method for colorimetric detection of a target analyte, the method comprising:
contacting one or more surfaces or solutions with a system comprising:
plasmonic gold nanoparticles (GNPs);
a binding receptor conjugated or adsorbed to a surface of the GNPs; and
a polymer-ligand conjugate; and
detecting emergence of a color wavelength or detecting a color change on the one or more surfaces or solutions, indicating existence of the target analyte.

21-36. (canceled)

37. A kit for colorimetric detection of a target analyte, the kit comprising:
a system comprising:
plasmonic gold nanoparticles (GNPs);
a binding receptor conjugated or adsorbed to a surface of the GNPs;
a polymer-ligand conjugate; and
one or more salts;
a system applicator;
optionally, a detection device;
optionally, one or more articles of personal protective equipment;
optionally, one or more buffers or receptacles; and
optionally, one or more of packaging or instructions for use.


SEQUENCE LISTING (1 sequence)

SEQ ID NO: 1 - protein, 288 aa, synthetic construct
SMITAIKIHEEYKKVCEEGKKGNKLVMEVIVRYTKMKTATNIYIFNLAKADALAESTLPFQSVNKLMGTWPFGTILCKKVISIDYYNMFTSIFTLCTMSVDRYIAVCHPVKALDFRTPRNAKEENEKNWKLSSEIGKPVEKKATTKYRQGSIDCTLTFSHPTWYWEDKLKDEVFKKAFEEPVKKIKECYGLMILRLKSVRMLSGSKEKDRNLRRITRMVLVVVEVFIKCWTEIHKYVKEGKLVTIPETTFQTVSWHECIAKGYKNSCENPKLYEELDENFKRCFREFC